SEQUENCE LISTING (6 sequences)

SEQ ID NO: 1 - DNA, 73 bp, Artificial Sequence
  Synthetic oligonucleotide control DNA
ttggtagtagtgattgtaatggtagtgatttttttttttttttcccccccccccccccccccccccccccccc

SEQ ID NO: 2 - DNA, 73 bp, Artificial Sequence
  Synthetic oligonucleotide aptamer.
ttggtggtggtggttgtggtggtggtggtttttttttttttttcccccccccccccccccccccccccccccc

SEQ ID NO: 3 - DNA, 30 bp, Artificial Sequence
  Synthetic oligonucleotide randomly generated sequence.
agtcacgtatacagctcatgatcagtcagt

SEQ ID NO: 4 - DNA, 30 bp, Artificial Sequence
  Synthetic oligonucleotide poly adenosine sequence
aaaaaaaaaaaaaaaaaaaaaaaaaaaaaa

SEQ ID NO: 5 - DNA, 30 bp, Artificial Sequence
  Synthetic oligonucleotide poly thymidine sequence
tttttttttttttttttttttttttttttt

SEQ ID NO: 6 - DNA, 30 bp, Artificial Sequence
  synthetic oligonucleotide poly cytosine sequence
cccccccccccccccccccccccccccccc

(19) United States
(12) Patent Application Publication
(10) Pub. No.: US 2012/0107242 A1
(43) Pub. Date: May 3, 2012

(54) NUCLEIC ACID-MEDIATED SHAPE CONTROL OF NANOPARTICLES FOR BIOMEDICAL APPLICATIONS

(75) Inventors: Zidong Wang, Urbana, IL (US); Yi Lu, Champaign, IL (US); Jieqian Zhang, Urbana, IL (US); Paul J. A. Kenis, Champaign, IL (US); Ngo Yin Wong, Urbana, IL (US)

(73) Assignee: The Board of Trustees of the University of Illinois

(21) Appl. No.: 13/249,070

(22) Filed: Sep. 29, 2011

Related U.S. Application Data
(60) Provisional application No. 61/404,410, filed on Sep. 30, 2010.

(51) Int. Cl. A61K 9/51; A61K 49/00; C12N 5/071; B32B 5/16; A61M 37/00; B05D 7/00; C12Q 1/02; A61K 31/7088; B82Y 5/00

(52) U.S. Cl. 424/9.1; 435/29; 435/375; 514/44 R; 424/493; 428/403; 604/20; 427/216; 427/214; 977/810; 977/902; 977/906

(57) ABSTRACT

Embodiments of a method for nucleic acid-mediated control of a nanoparticle shape are disclosed. In some embodiments, one or more nucleic acid oligomers are adsorbed to a metal nanoseed, and additional metal is deposited onto the nanoseed to produce a shaped nanoparticle. In certain embodiments, the nanoseed is gold and the oligomers are 5-100 nucleotides in length. The nanoparticle shape is determined at least in part by the nucleic acid sequence of the oligomer(s). Shaped nanoparticles produced by embodiments of the method include nanoflowers, nanospheres, nanostars, and nanoplates. Embodiments for using the shaped nanoparticles also are disclosed.

### CROSS REFERENCE TO RELATED APPLICATION

This application claims the benefit of the earlier filing date of U.S. Provisional Patent Application No. 61/404,410, filed Sep. 30, 2010, which application is incorporated herein by reference in its entirety.

### ACKNOWLEDGMENT OF GOVERNMENT SUPPORT

This invention was made with government support under Grant Nos. CMMI0749028, CTS0120978, and DMR0117792 awarded by the National Science Foundation. The government has certain rights in the invention.

### FIELD

Embodiments of a method for using nucleic acid molecules to control the growth and shape of nanoparticles are disclosed, as well nanoparticles and methods of using such nanoparticles.

### BACKGROUND

Metal nanoparticles have unique physicochemical properties leading to potential applications in selective catalysis, sensitive sensing, enhanced imaging, and medical treatment.^{1-9, 53, 54} The properties of a metal nanoparticle typically are affected by its size, shape, and crystal structure, and therefore it is possible to tune the properties of the particle by controlling its growth process. Molecular capping agents such as organic surfactants and polymers have been used to direct nanocrystal growth in a face-selective fashion to produce shape-controlled nanoparticle synthesis.^{8,9} Despite tremendous progress made, the mechanism of the shape control is not well understood, in part due to the difficulty in defining structures and conformations of these surfactants and polymers in solution and in systematic variation of functional groups.

DNA is a biopolymer with more defined structure and conformation in solution and unique programmable nature to tune its functional properties.¹⁰⁻¹³ Because of these advantages, DNA has been used as a template to position nanoparticles through DNA metallization,^{14,15} or nanoparticle attachment,¹⁶⁻²¹ or to control the sizes and/or the photo-luminescent properties of quantum dots.²²⁻²⁸ However, in contrast to proteins or peptides,²⁹⁻³² DNA has been much less explored to control the shape or morphology of metal nanoparticles, and, therefore the promise of this field remains to be fully realized. Such an investigation may result in new nanoparticles with new shapes and offer deeper insights into mechanisms of shape control.

### SUMMARY

Embodiments of a method to use DNA and/or RNA for modulating the shape and thus the optical properties of nanoparticles are disclosed. Systematic variations of the nucleic acid sequences offer mechanistic insights into the morphology control. Nucleic acid molecules in such nanoparticles maintain their bioactivity, allowing programmable assembly of new nanostructures. In addition, the cell uptake ability and light scattering property of the flower-shaped nanoparticles are also demonstrated. In some embodiments, the nucleic acid-mediated nanoparticle synthesis method is applied to synthesize non-spherical gold nanoparticles with new shapes by using other nanoseeds such as nanoprisms or nanorods.

Embodiments of a method for controlling the shape of a nanoparticle using nucleic acid (DNA and/or RNA) oligomers are disclosed. In some embodiments, the method includes providing a metal nanoseed, adsorbing a plurality of nucleic acid oligomers to the metal nanoseed, wherein each nucleic acid oligomer has a nucleic acid sequence, and depositing metal onto the metal nanoseed to produce a shaped nanoparticle, wherein the shaped nanoparticle has a shape determined at least in part by the nucleic acid sequence of the oligomer. In some embodiments, inorganic nanoseeds such as silica or metal oxide nanoseeds are used. Following adsorption of the nucleic acid oligomers to the inorganic nanoseed, additional inorganic material is deposited onto the nanoseed to produce a shaped nanoparticle.

In some embodiments, the metal nanoseed is gold. In certain embodiments, the metal nanoseed is coated with citrate before adsorbing the oligomer. In some embodiments, the metal nanoseed is a nanosphere, a nanorod, or a nanoprism. In particular embodiments, the metal nanoseed has a largest dimension ranging from 1 nm to 1000 nm, such as from 1 nm to 25 nm, 1 nm to 50 nm, 1 nm to 100 nm, 1 nm to 250 nm, 1 nm to 500 nm, 5 nm to 20 nm, 5 nm to 50 nm, 5 nm to 100 nm, 5 nm to 150 nm, 10 nm to 50 nm, 10 nm to 100 nm, 10 nm to 500 nm, 10 nm to 1000 nm.

In some embodiments, each nucleic acid oligomer has a DNA sequence selected from poly A, poly C, poly G, poly T, or a sequence with mixed nucleotide of A, C, G, and/or T. In other embodiments, the oligomer is an RNA oligomer, and the RNA sequence is poly A, poly C, poly G, poly U, or a sequence with mixed nucleotides of A, C, G, and/or U. In some embodiments, the oligomer is an aptamer. In certain embodiments, the oligomer has at least 5 nucleotides, such as at least 10, at least 50, or at least 100 nucleotides, such as 5 to 100 nucleotides. In certain embodiments, the oligomer is labeled with a detectable label. In some embodiments, a plurality of oligomers is adsorbed to the metal nanoseed. In particular embodiments, the sequence of each of the plurality of oligomers is the same.

In some embodiments, the metal nanoseed is a gold nanosphere, a plurality of DNA oligomers is adsorbed to the gold nanosphere, wherein each of the plurality of DNA oligomers has a DNA sequence consisting of poly A, poly C, or a mixture of A and C, and depositing gold onto the gold nanosphere produces a nanoflower. In other embodiments, each of the plurality of DNA oligomers has a DNA sequence consisting of poly T, and depositing gold onto the gold nanosphere produces a spherical nanoparticle.

In some embodiments, the metal nanoseed is a gold nanoprism, a plurality of DNA oligomers are adsorbed to the gold nanoprism, wherein each of the plurality of DNA oligomers has a DNA sequence consisting of poly T or a mixture of T in majority and C in minority, and depositing gold onto the gold nanoprism produces a six-angled nanostar. In some embodiments, each of the plurality of DNA oligomers has a DNA sequence consisting of poly G, or a mixture of G in majority and T in minority, and depositing gold onto the gold nanoprism produces a nanostar with multiple tips. In other embodiments, each of the plurality of DNA oligomers has a DNA sequence consisting of poly A, poly C, or a mixture of A and C, and depositing gold onto the gold nanoprism produces a nanoplate.

Also disclosed are embodiments of shaped nanoparticles including a metal nanoparticle and a plurality of oligomers extending from the metal nanoparticle, wherein at a least a portion of each of the plurality of oligomers is embedded within the metal nanoparticle. In some embodiments, the oligomers are at least 5 nucleotides, such as at least 10, at least 50, or at least 100 nucleotides, such as 5 to 100 nucleotides in length. In particular embodiments, the metal nanoparticle is gold.

In some embodiments, the metal nanoparticle is gold, the oligomers are DNA oligomers that are at least 5 nucleotides, such as at least 10, at least 50, or at least 100 nucleotides, such as 5 to 100 nucleotides in length, each of the DNA oligomers has a DNA sequence consisting of poly A, poly C, or a mixture of A and C, and the shaped nanoparticle is a nanoflower or a nanoplate. In other embodiments, each of the DNA oligomers has a DNA sequence consisting of poly T, poly G or a mixture of T and G, and the shaped nanoparticle is a nanosphere or a nanostar.

In some embodiments, the oligomers are RNA oligomers that are at least 5 nucleotides, such as at least 10, at least 50, or at least 100 nucleotides, such as 5 to 100 nucleotides in length, and each of the RNA oligomers has an RNA sequence consisting of poly A, poly C, poly G, poly U, or a mixture of A, C, G, and/or U.

Embodiments of methods of using the shaped nanoparticles also are disclosed. In some embodiments, the shaped nanoparticle is delivered to a target cell by contacting the shaped nanoparticle with a target cell under conditions that allow the shaped nanoparticle to enter or bind to the cell. In certain embodiments, the shaped nanoparticle is conjugated to an antibody specific for a protein on the surface of the target cell, thereby delivering the shaped nanoparticle to the target cell. In particular embodiments, the shaped nanoparticle comprises oligomers including an aptamer sequence extending from the shaped nanoparticle, wherein the aptamer sequence is capable of binding to the target cell (e.g., to a protein on the surface of the target cell), thereby delivering the shaped nanoparticle to the target cell. In certain embodiments, the target cell is in a subject, and contacting comprises administering the shaped nanoparticle to the subject.

Embodiments of methods of using the shaped nanoparticles also are disclosed. In some embodiments, the shaped nanoparticle is delivered to a target cell by contacting the shaped nanoparticle with a target cell under conditions that allow the shaped nanoparticle to bind to and/or enter the cell, wherein the shaped nanoparticle comprises DNA or RNA aptamers specific for the target cell, thereby delivering the shaped nanoparticle to a target cell. In certain embodiments, the target cell is in a subject, and contacting comprises administering the shaped nanoparticle to the subject.

In some embodiments, the shaped nanoparticle is imaged after delivery to the target cell. In other embodiments, after the shaped nanoparticle is delivered to the target cell in the subject, near-infrared radiation is administered to the subject, wherein the shaped nanoparticle absorbs at least a portion of the near-infrared radiation, thereby producing a temperature increase within the shaped nanoparticle.

In some embodiments, a drug is delivered within a cell by contacting an embodiment of a shaped nanoparticle with the cell, wherein the shaped nanoparticle comprises a drug molecule conjugated to the shaped nanoparticle to produce a drug-shaped nanoparticle conjugate, and wherein the drug-shaped nanoparticle conjugate is contacted with the cell under conditions sufficient to allow the cell to bind to and/or internalize the drug-shaped nanoparticle conjugate. In certain embodiments, the cell is in a subject, and contacting comprises administering a therapeutic amount of the drug-shaped nanoparticle to the subject.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1a depicts UV-visible spectra of gold nanoparticle solutions prepared with A30 oligomers (AuNF_A30, dark blue line), C30 oligomers (AuNF_C30, blue line), T30 oligomers (AuNF_T30, red line), in the absence of DNA (AuNF_No DNA, pink line), or before reduction (AuNS/No reduction, light pink line); FIG. 1b is a series of color photographs of the corresponding gold nanoparticles.

FIGS. 2a-d are a series of transmission electron microscopy (TEM) images of gold nanoparticles prepared with (a) A30 oligomers, (b), C30 oligomers, (c) T30 oligomers, (d) in the absence of DNA. The scale bar indicates 20 nm.

FIG. 3 is a TEM image of gold nanoparticles prepared with G10 oligomers. The scale bar indicates 20 nm.

FIG. 4a is a TEM image of 200-nm gold nanoseeds (AuNS).

FIG. 4b is a TEM image of gold nanoparticles prepared in the absence of DNA but with the addition of 20 mM NaCl. It is noted that aggregation of the gold nanoparticles occurred during synthesis.

FIGS. 5a-5d are color photographs of AuNS solutions incubated with (a) A30 oligomers, (b) C30 oligomers, (c) T30 oligomers, and (d) in the absence of DNA before (left image of each pair) and after (right image of each pair) the addition of 0.1 M NaCl.

FIG. 5e is a series of UV-visible spectra of the corresponding nanoparticle solutions with and without the presence of 0.1 M NaCl.

FIGS. 6a-f are TEM images of gold nanoparticles prepared by reducing (a) 0.05 μL, (b) 0.1 μL, (c) 0.4 μL, (d) 0.6 μL, (e) 1.2 μL, and (f) 2.0 μL of 1% HAuCl₄ aqueous solution with an excess amount of NH₂OH (20 mM). Before the reduction reaction, 100 μL of 0.5 nM AuNS solution was incubated with 1 μM poly A30. The scale bar indicates 20 nm.

FIGS. 7a-f are TEM images of gold nanoparticles prepared by incubating AuNS solutions with poly A30 at different molar ratios: AuNS:DNA=(a) 1:20, (b) 1:100, (c) 1:500, (d) 1:1000, (e) 1:2000, (f) 1:4000. The AuNS solutions (0.5 nM) were incubated with DNA for 30 minutes, followed by addition of 20 mM NH₂OH and 167 μM HAuCl₄ to complete the nanoparticle synthesis. The scale bar indicates 20 nm.

FIGS. 8a-b are TEM images of gold nanoparticles prepared with (a) adenosine monophosphate (AMP), and (b) random 30-mer DNA. A similar synthesis procedure was followed except that 0.5 nM AuNS was incubated with 30 μM AMP or 1 μM random DNA with the sequence 5′-AGT CAC GTA TAC AGC TCA TGA TCA GTC AGT-3′ (SEQ ID NO: 3). The scale bar indicates 20 nm.

FIG. 9 depicts the time-dependent evolution of the UV-visible spectra of gold nanoflowers (AuNF) grown in the presence of A30 oligomers. From bottom to top, the spectra illustrate the absorbance of the growth solution after initiation of the reaction for 0 s, 3 s, 5 s, 10 s, 30 s, 60 s, 120 s, 240 s, 480 s, 720 s, and 840 s, respectively.

FIGS. 10a-r are TEM images of the nanoparticle intermediates prepared by stopping the nanoparticle growth with mercaptopropionic acid (1.5 mM) after 0.5 s (a, g, m), 2 s (b, h, n), 5 s (c, i, o), 30 s (d, j, p), 5 min. (e, k, q) and 15 min. (f, l, r) of the reaction. The images in the top row (a-f) represent the intermediates synthesized in the presence of poly A30 oligomers; the images in the second row (g-l) represent the intermediates synthesized in the presence of poly T30 oligomers; the images in the last row (m-r) represent the intermediates synthesized in the absence of DNA. Before initiation of the reduction reaction, 100 μL of 0.5 nM AuNS solution was incubated with 1 μM DNA. The scale bar indicates 20 nm.

FIG. 11 is a TEM image of small gold nanoparticles produced from the conversion of Au(I)-mercaptopropionic acid complexes into metal particles on the TEM grid upon electron-beam irradiation during TEM imaging. HAuCl₄ (167 μM) was mixed with mercaptopropionic acid (1.5 mM), and the mixture was dropped on the TEM grid. The TEM image was taken after the sample was dried. The scale bar indicates 20 nm.

FIG. 12 is a schematic illustration of one embodiment of a method for DNA-mediated shape control of gold nanoparticles. Poly A (SEQ ID NO: 4); Poly T (SEQ ID NO: 5); Poly C (SEQ ID NO: 6).

FIG. 13 depicts melting curves of the DNA on AuNFs (circles) and free DNA in solution (squares). Both melting curves were obtained using buffer containing 10 mM HEPES buffer (pH 7.1) and 50 mM NaCl.

FIGS. 14a-d are TEM images of nanoassemblies: (a) AuNF_A30 with AuNS₅ₙₘ_{_{—}}S_T30; (b) AuNF_A30 with non-complementary AuNS₅ₙₘ_{_{—}}S_A30; (c) AuNS_T30 with AuNS₅ₙₘ_{_{—}}S_A30; (d) AuNS_T30 with non-complementary AuNS₅ₙₘ_{_{—}}S_T30. The scale bar indicates 20 nm.

FIGS. 15a-d are TEM images of nanoassemblies: (a, b) AuNF_A30 with AuNS₅ₙₘ_{_{—}}S_T30; (c, d) AuNF_A30 with non-complementary AuNS₅ₙₘ_{_{—}}S_A30. The scale bar indicates 100 nm.

FIG. 16 depicts Raman spectra of the Raman tag (Trama) from AuNFs (upper line) and AuNSs (lower line). The samples were excited with 603 nm laser.

FIG. 17 is a dark-field light-scattering image of gold nanoflowers. The scale bar indicates 2 μm.

FIGS. 18a-b are dark-field images of Chinese hamster ovary (CHO) cells (a) treated with AuNF particles, (b) without nanoparticle treatment. The scale bar indicates 10 μm.

FIGS. 19a-h are optical and confocal fluorescence images of CHO cells treated with AuNF nanoparticles synthesized with FAM-A30 (a-d) or without nanoparticle treatment (e-h). FIG. 19a is a brightfield image of the AuNF treated cells; FIGS. 19b-d are corresponding 3-D reconstructed confocal fluorescence images of the AuNF treated cells (b: top view; c, d: side views; unit scale: 1 μm); FIG. 19e is a brightfield image of the control cells; FIGS. 19f-h are corresponding 3-D reconstructed confocal fluorescence images of the control cells (f: top view; g, h: side views; unit scale: 1 μm). The scale bars in FIGS. 19a and 19e indicate 10 μm. The AuNFs (1 nM) were incubated with CHO cells for 20 hours before imaging. The fluorescence arises from the incomplete quenching of fluorophore by the gold nanoparticles. It was shown that the fluorescent dots were distributed inside the cells, indicating that the AuNFs were taken up by the cells after incubation. As a comparison, the control cells without nanoparticle treatment showed little fluorescence.

FIGS. 20a-d are TEM images of nanoparticles synthesized with A30 oligomers (a), T30 oligomers (b), C30 oligomers (c) and G10 oligomers (d) by using gold nanoprisms as seeds.

FIGS. 21a-c are TEM images of nanorod seeds before reaction (a), and nanoparticles synthesized with A30 oligomers (b), and T30 oligomers (c) using the gold nanorod seeds.

FIGS. 22a-d are TEM images of nanoflowers synthesized with increasing concentrations of gold.

FIGS. 23a-b are graphs of size versus gold salt concentration, demonstrating a linear relationship between gold salt concentration and nanoflower size. The nanoflowers were synthesized with a randomized DNA construct (a) or an AS1411 aptamer (b); 50 particles were counted to determine size.

FIGS. 24a-24c are TEM images of gold nanoflowers grown from 15-nm, 30-nm, and 50-nm gold nanoparticles, respectively.

FIG. 25 is a graph illustrating the absorption spectra of gold nanoflowers grown from 15-nm, 30-nm, and 50-nm gold nanoparticles.

FIGS. 26a-26b are dark-field optical images of MCF-7 cells incubated with nanoflowers comprising control DNA (a) or nanoflowers comprising the AS1411 aptamer (b). The images were obtained under identical conditions and microscope settings.

### SEQUENCE LISTING

The nucleic acid sequences provided herein are shown using standard letter abbreviations for nucleotide bases as defined in 37 C.F.R. 1.822. Only one strand of each nucleic acid sequence is shown, but the complementary strand is understood as included by any reference to the displayed strand. The sequence listing is submitted as an ASCII text file, named 7950-85921-02_ST25.txt,” created on Sep. 27, 2011, 2011, 1.83 KB, which is incorporated by reference herein.

SEQ ID NO: 1 is a randomized control DNA sequence.

SEQ ID NO: 2 is a DNA sequence including the AS1411 aptamer sequence.

SEQ ID NO: 3 is a randomized DNA sequence.

SEQ ID NO: 4 is a Poly A sequence.

SEQ ID NO: 5 is a Poly T sequence.

SEQ ID NO: 6 is a Poly C sequence.

### DETAILED DESCRIPTION

Embodiments of a method for using nucleic acids to control nanoparticle shape are disclosed. The nucleic acids may be DNA or RNA. Single strand DNA (ssDNA) has been found to adsorb on citrate-coated gold nanospheres (AuNSs) in a sequence-dependent manner.³³ Deoxynucleosides dA, dC, dG have shown much higher binding affinity to gold surfaces than deoxynucleoside dT.³⁴ To investigate the effect of different DNA sequences on nanoparticle morphology during crystal growth, various DNA oligomers were bound to gold nanoseeds, additional metal was deposited onto the DNA-nanoseed constructs, and the resulting nanoparticle morphology was determined.

Nanoparticles made by some embodiments of the disclosed method can be taken up by cells. Because metallic nanoparticles can be visualized by, e.g., darkfield microscopy, such nanoparticles may be useful for intracellular imaging. Additionally, nanoparticles that can be taken up by cells may be useful carriers for delivering drugs, contrast agents, genes, and other molecules into cells.

### I. TERMS AND ABBREVIATIONS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.”Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Definitions of common terms in chemistry may be found in Richard J. Lewis, Sr. (ed.), Hawley's Condensed Chemical Dictionary, published by John Wiley & Sons, Inc., 1997 (ISBN 0-471-29205-2). All references herein are incorporated by reference. In order to facilitate review of the various embodiments of the disclosure, the following explanations of specific terms are provided:

Administration: To provide or give a subject an agent, such as a nanoparticle preparation described herein, by any effective route. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes.

Adsorption: The physical adherence or bonding of ions and molecules onto the surface of another molecule or substrate. An ion or molecule that adsorbs is referred to as an adsorbate. Adsorption can be characterized as chemisorption or physisorption, depending on the character and strength of the bond between the adsorbate and the substrate surface. Chemisorption is characterized by a strong interaction between an adsorbate and a substrate, e.g., formation of covalent and/or ionic bonds. Physisorption is characterized by weaker bonding between an adsorbate and a substrate. The weaker bond typically results from van der Waals forces, i.e., an induced dipole moment between the adsorbate and the substrate.

Antibody: A polypeptide ligand comprising at least a light chain or heavy chain immunoglobulin variable region which specifically recognizes and binds an epitope of an antigen, such as a tumor-specific protein. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

Aptamer: An oligonucleic acid that binds to a specific target. Nucleic acid aptamers are capable of binding to various molecular targets such as small molecules, proteins, nucleic acids, or cells. DNA or RNA aptamers recognize target effector molecules with high affinity and specificity (Ellington and Szostak, Nature 346(6287):818-822, 1990; Tuerk and Gold, Science, 249:505-510, 1990). Aptamers have several unique properties. First, aptamers for a given target can be obtained by routine experimentation. For instance, in vitro selection methods can be used (called systematic evolution of ligands by exponential enrichment (SELEX)) to obtain aptamers for a wide range of target effector molecules with exceptionally high affinity, having dissociation constants in the picomolar range (Brody and Gold, Reviews in Molecular Biotechnology, 74(1)5-13, 2000, Jayasena, Clinical Chemistry, 45(9):1628-1650, 1999, Wilson and Szostak, Ann. Rev. Biochem., 68:611-647, 1999, Ellington et al., Nature 1990, 346, 818-822; Tuerk and Gold Science 1990, 249, 505-510; Liu et al., Chem. Rev. 2009, 109, 1948-1998; Shamah et al., Acc. Chem. Res. 2008, 41, 130-138; Famulok, et al., Chem. Rev. 2007, 107, 3715-3743; Manimala et al., Recent Dev. Nucleic Acids Res. 2004, 1, 207-231; Famulok et al., Acc. Chem. Res. 2000, 33, 591-599; Hesselberth, et al., Rev. Mol. Biotech. 2000, 74, 15-25; Morris et al., Proc. Natl. Acad. Sci. U.S.A. 1998, 95, 2902-2907). Second, aptamers are easier to obtain and less expensive to produce than antibodies, because aptamers can be generated in vitro in short time periods (for example, within days) and at economical cost. Third, aptamers display remarkable structural durability and can be denatured and renatured many times without losing their ability to recognize their targets. The mononucleotides of an aptamer may adopt a particular conformation upon binding to its target. Aptamers that are specific to a wide range of targets from small organic molecules such as adenosine, to proteins such as thrombin, and even viruses and cells, have been identified (Chou et al., Trends in Biochem Sci. 2005, 30(5), 231-234; Liu et al., Chem. Rev. 2009, 109, 1948-1998; Lee et al., Nucleic Acids Res. 2004, 32, D95-D100; Navani and Li, Curr. Opin. Chem. Biol. 2006, 10, 272-281; Song et al., TrAC, Trends Anal. Chem. 2008, 27, 108-117; Tombelli et al., Bioelectrochemistry, 2005, 67(2), 135-141). In one example the aptamer is specific for HIV (such as HIV-tat).

Contacting: Placement in direct physical association, including both a solid and liquid form. Contacting can occur in vitro, for example, with isolated cells, such as tumor cells, or in vivo by administering to a subject (such as a subject with a tumor). Thus, the nanoparticles disclosed herein can be contacted with cells in vivo or in vitro, under conditions that permit the nanoparticle to be endocytosed into the cell.

DNA melting temperature: The temperature at which a DNA double helix dissociates into single strands, specifically the temperature at which 50% of the DNA, or oligonucleotide, is in the form of a double helix and 50% has dissociated into single strands. The most reliable and accurate determination of melting temperature is determined empirically. Methods for determining the melting temperature of DNA are known to those with ordinary skill in the art of DNA characterization. For single-stranded oligomers, a complementary oligonucleotide is hybridized to the oligomer, and the melting temperature of the double-stranded complex is determined.

Nanoflower (NF): A nanoparticle with a morphology in microscopic view that resembles a flower.

Nanoparticle (NP): A nanoscale particle with a size that is measured in nanometers, for example, a particle that has at least one dimension of less than about 100 nm. Nanoparticles may have different shapes, e.g., nanofibers, nanoflowers, nanohorns, nano-onions, nanopeanuts, nanoplates, nanoprisms, nanorods, nanoropes, nanospheres, nanostars, nanotubes, etc.

Nanoplate: A nanoparticle with a morphology in microscopic view that resembles a substantially flat plate.

Nanoseed (NS): A small nanoparticle used as a starting material for larger nanoparticle synthesis. For example, gold ions may be reduced and deposited onto gold nanoseeds to produce larger gold nanoparticles.

Nanostar: A nanoparticle with a morphology in microscopic view that resembles a star.

Near-infrared (NIR): The infrared spectrum is typically divided into three sections, with near-infrared including the shortest wavelengths. Although the region is not rigidly defined, NIR typically encompasses light with wavelengths ranging from 700-2000 nm.

An oligomer is a general term for a polymeric molecule consisting of relatively few monomers, e.g., 5-100 monomers. In one example, the monomers are nucleotides.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of the nanoparticles disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A polymer is a molecule of repeating structural units (e.g., monomers) formed via a chemical reaction, e.g., polymerization.

“Specifically binds” refers to the ability of a molecule to bind with specificity to a particular target. For example, “specifically binds” refers to the ability of an individual aptamer to specifically bind to a molecular target such as a small molecule, a protein, a particular nucleic acid sequence, or a particular cell.

“Specifically binds” also refers to the ability of individual antibodies to specifically immunoreact with an antigen, such as a tumor-specific antigen, relative to binding to unrelated proteins, such as non-tumor proteins, for example β-actin. For example, a HER2-specific binding agent binds substantially only the HER-2 protein in vitro or in vivo. As used herein, the term “tumor-specific binding agent” includes tumor-specific antibodies and other agents that bind substantially only to a tumor-specific protein in that preparation.

The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody”.

In some examples, an antibody (such as an antibody conjugated to a nanoparticle of the present disclosure) specifically binds to a target (such as a cell surface protein) with a binding constant that is at least 10³ M⁻¹ greater, 10⁴M⁻¹ greater or 10⁵ M⁻¹ greater than a binding constant for other molecules in a sample or subject. In some examples, an antibody (e.g., monoclonal antibody) or fragments thereof, has an equilibrium constant (Kd) of 1 nM or less. For example, an antibody binds to a target, such as tumor-specific protein with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, N.J.

Subject or patient: A term that includes human and non-human mammals. In one example, the subject is a human or veterinary subject, such as a mouse.

Therapeutically effective amount: An amount of a composition that alone, or together with an additional therapeutic agent(s) (such as a chemotherapeutic agent) sufficient to achieve a desired effect in a subject, or in a cell, being treated with the agent. The effective amount of the agent (such as the nanoparticles disclosed herein) can be dependent on several factors, including, but not limited to the subject or cells being treated, the particular therapeutic agent, and the manner of administration of the therapeutic composition. In one example, a therapeutically effective amount or concentration is one that is sufficient to prevent advancement, delay progression, or to cause regression of a disease, or which is capable of reducing symptoms caused by the disease, such as cancer. In one example, a therapeutically effective amount or concentration is one that is sufficient to increase the survival time of a patient with a tumor.

In one example, a desired response is to reduce or inhibit one or more symptoms associated with cancer. The one or more symptoms do not have to be completely eliminated for the composition to be effective. For example, administration of a composition containing a nanoparticle disclosed herein, which in some examples is followed by photothermal therapy can decrease the size of a tumor (such as the volume or weight of a tumor, or metastasis of a tumor) by a desired amount, for example by at least 20%, at least 50%, at least 80%, at least 90%, at least 95%, at least 98%, or even at least 100%, as compared to the tumor size in the absence of the nanoparticle. In one particular example, a desired response is to kill a population of cells by a desired amount, for example by killing at least 20%, at least 50%, at least 60%, at least 70%, at least 80%, at least 90%, at least 95%, at least 98%, or even at least 100% of the cells, as compared to the cell killing in the absence of the nanoparticle. In one particular example, a desired response is to increase the survival time of a patient with a tumor (or who has had a tumor recently removed) by a desired amount, for example increase survival by at least 20%, at least 50%, at least 60%, at least 70%, at least 80%, at least 90%, at least 95%, at least 98%, or even at least 100%, as compared to the survival time in the absence of the nanoparticle.

The effective amount of the disclosed nanoparticles that is administered to a human or veterinary subject will vary significantly depending upon a number of factors associated with that subject, for example the overall health of the subject. An effective amount of an agent can be determined by varying the dosage of the product and measuring the resulting therapeutic response, such as the regression of a tumor. Effective amounts also can be determined through various in vitro, in vivo or in situ immunoassays. The disclosed agents can be administered in a single dose, or in several doses, as needed to obtain the desired response. However, the effective amount of the disclosed nanoparticles can be dependent on the source applied, the subject being treated, the severity and type of the condition being treated, and the manner of administration. In certain examples, a therapeutically effective dose of the disclosed nanoparticles is at least 20 mg per kg body weight, at least 200 mg per kg, at least 2,000 mg per kg, or at least 20 g per kg, for example when administered intravenously (iv).

In particular examples, a therapeutically effective dose of an antibody conjugated to a nanoparticle of the present disclosure is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, for example 0.5 to 50 mg/60 kg, such as a dose of 1 mg/60 kg, 2 mg/60 kg, 5 mg/60 kg, 20 mg/60 kg, or 50 mg/60 kg, for example when administered iv. However, one skilled in the art will recognize that higher or lower dosages also could be used, for example depending on the particular nanoparticle. In particular examples, such daily dosages are administered in one or more divided doses (such as 2, 3, or 4 doses) or in a single formulation. The disclosed nanoparticle can be administered alone, in the presence of a pharmaceutically acceptable carrier, in the presence of other therapeutic agents (such as other anti-neoplastic agents).

Treating: A term when used to refer to the treatment of a cell or tissue with a therapeutic agent, includes contacting or incubating an agent (such as a nanoparticle disclosed herein) with the cell or tissue. A treated cell is a cell that has been contacted with a desired composition in an amount and under conditions sufficient for the desired response. In one example, a treated cell is a cell that has been exposed to a nanoparticle under conditions sufficient for the nanoparticle to enter the cell, which is in some examples followed by phototherapy, until sufficient cell killing is achieved.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated with the claimed nanoparticles include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma.

The disclosed nanoparticles can also be used to treat liquid tumors, such as a lymphatic, white blood cell, or other type of leukemia.

Under conditions sufficient for: A phrase that is used to describe any environment that permits the desired activity. In one example, “under conditions sufficient for” includes administering a nanoparticle to a subject sufficient to allow the nanoparticle to enter the cell. In particular examples, the desired activity is killing the cells into which the nanoparticles entered, for example following phototherapy of the cells. In another example, “under conditions sufficient for” includes contacting DNA oligomers with a nanoseed sufficient to allow the oligomers to bind to the nanoseed, to form a nanoparticle of the desired shape.

### II. NANOPARTICLE PREPARATION AND NUCLEIC ACID-MEDIATED SHAPE CONTROL

The disclosure provides nanoparticles having attached thereto nucleic acid oligomers, wherein the DNA or RNA oligomer can be used to control the shape of the nanoparticle. Also provided are methods of making such shaped nanoparticles.

In some embodiments, nanospheres (NSs) are used as nanoseeds, or starting materials, for nanoparticle growth. In other embodiments, the nanoseeds are nanoprisms, or nanorods. A person of ordinary skill in the art of nanoparticle technology will understand that nanoseeds of any shape may be used; however, the final nanoparticle's morphology may depend at least in part upon the shape of the nanoseed. Nanoseeds may comprise any material to which nucleic acid oligomers can be attached. If the nanoparticles will be administered to living subjects, it is advantageous to use nanoseeds that do not have significant cellular toxicity. In particular embodiments, gold nanoparticles are produced from gold nanoseeds. Gold has very low cellular toxicity, making gold nanoparticles (NPs) advantageous for applications in living subjects. Other suitable materials include other metals, such as silver and platinum, as well as inorganic compounds (e.g., silica, metal oxide).

Typically, the nanoseeds have a largest dimension, or diameter, between 1 nm to 1000 nm, such as from 1 nm to 25 nm, 1 nm to 50 nm, 1 nm to 100 nm, 1 nm to 250 nm, 1 nm to 500 nm, 5 nm to 20 nm, 5 nm to 50 nm, 5 nm to 100 nm, 5 nm to 150 nm, 10 nm to 50 nm, 10 nm to 100 nm, 10 nm to 500 nm, 10 nm to 1000 nm. In some embodiments, AuNSs with a diameter of 5-20 nm were used.

In some embodiments, nucleic acid oligomers comprising a single type of nucleotide are used (e.g., poly A). In other embodiments, the oligomers may include more than one type of nucleotide (such as an oligomer containing a mixture of A and C). Oligomers containing five or more nucleotides are suitable for use in the disclosed embodiments. Oligomers with fewer than 5 nucleotides are too short to significantly influence the nanoparticle morphology. The oligomers disclosed herein can be at least 5 nucleotides in length, such as at least 10, at least 20, at least 30, or at least 60 nucleotides in length, such as 5 to 100 nucleotides in length, 5 to 60 nucleotides, or 10 to 30 nucleotides in length. In some embodiments, all of the oligomers bound to the NS have the same sequence and the same length. In other embodiments, oligomers of differing sequences and/or differing lengths may be used. In a working embodiment, 30-mer DNAs consisting of either poly A, poly C, or poly T (designated as A30, C30, and T30, respectively) were bound to AuNSs.

In some embodiments, the oligomers may be modified or labeled with a detectable label. Suitable detectable labels may include, but are not limited to, fluorophores (e.g., fluorescein dyes, Alexa Fluor® dyes, etc.), radioisotopes, biotin, photo-sensitive linkers, and chemical functional groups (e.g., alkynyl, azide, carboxyl, etc.).

In some embodiments, gold nanoseeds are coated with citrate during nanoseed synthesis. Oligomers adsorb to the citrate-coated AuNS surface via physisorption.

After nucleic acid (NA) oligomers are adhered to the NS surface, additional material is deposited onto the nucleic acid-nanoseed (NA-NS) construct to produce nanoparticle growth. In some embodiments, the nanoseed is gold, and nanoparticle growth is achieved through gold ion reduction and deposition onto the NA-functionalized AuNS surface. In working embodiments, hydrogen tetrachloroaurate(III) (HAuCl₄) was used as the gold ion source. However, other soluble gold salts also may be used. Hydroxylamine (NH₂OH) is a suitable reducing agent for reducing HAuCl₄ catalyzed by the gold surface.³⁵ Other reducing agents also may be used, e.g., ascorbic acid, amines (poly(allylamine) hydrochloride⁵¹, sodium diphenylamine sulfonate⁵²).

Nanoparticle size is controlled by varying the size of the nanoseed and/or varying the growth conditions. In some embodiments, a set of growth conditions is selected to minimize the amount of gold deposited onto the nanoseed. For example, the amount of HAuCl₄ can be limited and controlled to precisely control the size of the resulting nanoparticle. In certain embodiments, the nanoparticle has a largest dimension, or diameter of 5-1,000 nm, such as 10-500 nm, 10-250 nm, or 20-200 nm. Typically, particles with a largest dimension between 20 nm and 200 nm are suitable for in vivo applications. Nanoparticle size is a result-effective variable that may influence uptake activity for nanoparticles having a particular shape, surface functionalization, and/or environment.

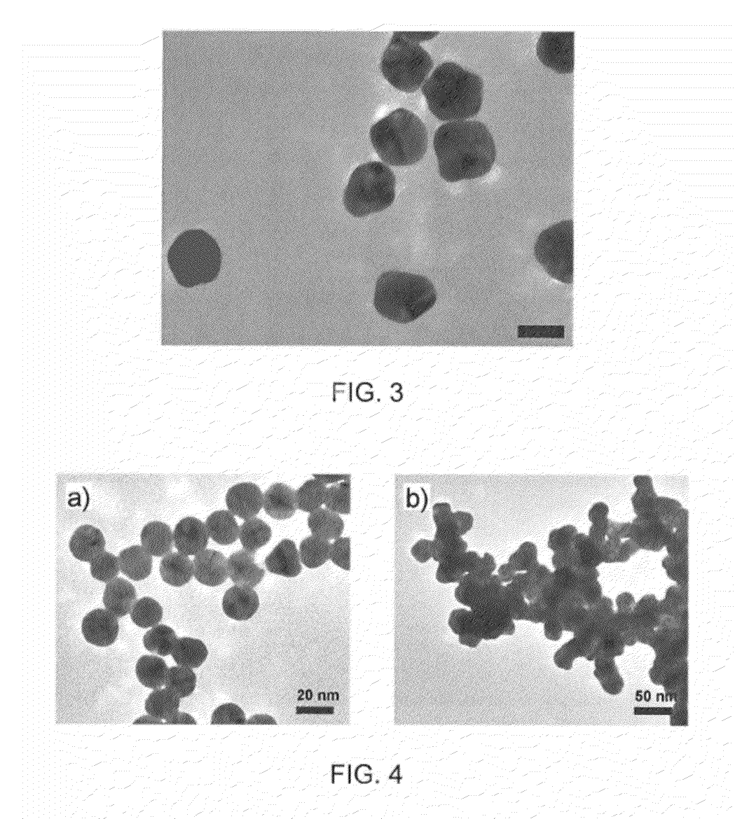

The oligomer sequence affects the morphology of the nanoparticle. In a working embodiment, gold nanoparticles were synthesized in the presence of A30, C30, or T30 oligomers. DNA oligomers were adsorbed onto small gold nanospheres. Gold ions in solution subsequently were reduced and deposited onto the DNA-nanosphere constructs to cause nanoparticle growth. Using transmission electron microscopy to determine the nanoparticles' morphology, the inventors unexpectedly discovered that the nanoparticles synthesized in the presence of A30 and C30 were flower shaped (FIGS. 2a-2b), while nanoparticles synthesized in the presence of T30 were spherical (FIG. 2c). Nanoparticles synthesized in the absence of DNA also were spherical (FIG. 2d), as were nanoparticles synthesized in the presence of a 10-mer of poly G (FIG. 3). Thus, it is apparent that the nucleic acid sequence mediated the nanoparticle growth and controlled the resulting shape of the nanoparticle.

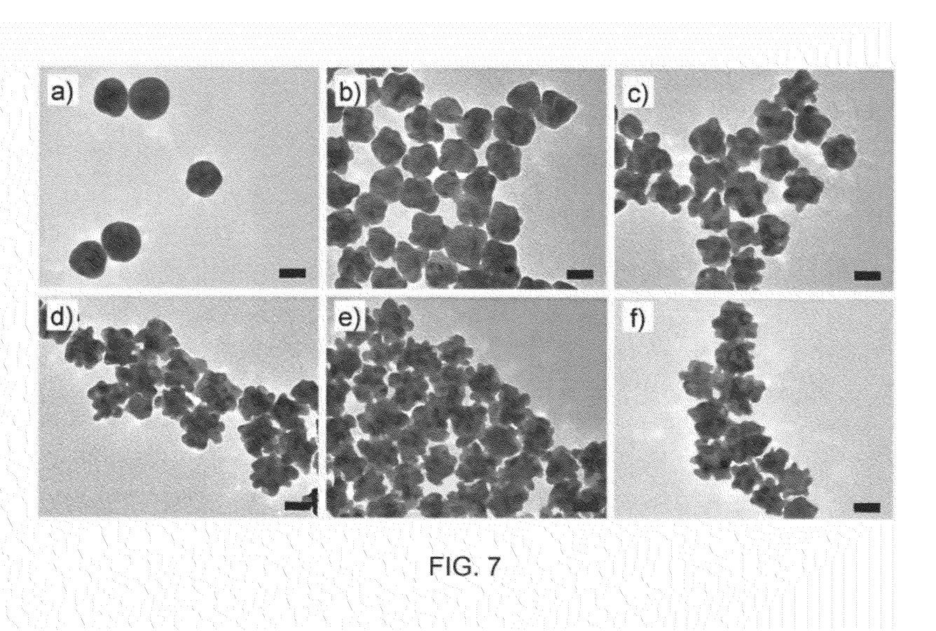

The length and number of oligomers adsorbed to the nanoseed also significantly affect the nanoparticle shape. As previously discussed, shorter oligomers (e.g., those with fewer than 5 nucleotides) have a lesser influence on the nanoparticle shape. Furthermore, the number of oligomers adsorbed to the nanoseed significantly affects the nanoparticle morphology. As shown in FIGS. 7a-f, shape control becomes increasingly evident as the number of oligomers increases.

Thiol chemistry can be used to conjugate DNA and RNA to gold surfaces. However, when thiolated (i.e., thiol-modified) DNA is adsorbed onto gold nanospheres, all of the thiolated DNA can be displaced by mercaptoethanol. In contrast, embodiments of AuNFs produced with unmodified poly A oligonucleotides by the methods disclosed herein are resistant to mercaptoethanol displacement, and incubation with mercaptoethanol overnight displaces less than one-third of the DNA strands. Thus, some embodiments of the disclosed in situ synthesis and controlled reduction methods advantageously can be used to prepare stable DNA-functionalized gold surfaces with unmodified DNA. Certain embodiments of gold nanoflowers produced by the disclosed methods are very stable in aqueous solution, even in the presence of 0.3 M salt, demonstrating that unmodified DNA oligomers can be attached to the nanoparticles during their synthesis, and act as stabilizing ligands.

Considering the remarkably high binding affinity of DNA to the AuNFs (higher than thiol-gold binding), it was hypothesized that the DNA in situ attached to AuNFs during reduction could be partially buried in the AuNFs. As additional gold is deposited onto the DNA-functionalized nanoseed, a portion of the DNA strand becomes buried in the deposited gold, thereby firmly attaching the DNA oligomer to the nanoparticle during nanoparticle growth. Because the melting point of a DNA oligonucleotide bound to a complementary oligonucleotide increases with the length of the oligonucleotide, an attached DNA oligonucleotide may have a lower melting point than that of a free oligonucleotide if a portion of the attached oligonucleotide is buried within the gold nanoparticle. In some embodiments, the attached oligonucleotides have a melting point that is at least 10% or at least 20% (such as 10-20%) lower than that of the corresponding free oligonucleotides, substantiating the hypothesis that a portion of the DNA strand is embedded within the gold nanoparticle during nanoparticle growth. In certain embodiments, it is preferable to control nanoparticle size by varying the nanoseed size rather than by varying the thickness of the deposited gold. Varying the nanoseed size while minimizing the thickness of the deposited gold allows minimal “trapping” of the DNA sequence by the growing gold layer.

To produce flower-shaped gold nanoparticles, the DNA oligomer has at least 5 nucleotides. DNA oligomers with fewer nucleotides are not long enough to significantly influence the nanoparticle morphology. As discussed above, DNA oligomers comprising poly A and poly C produced flower-shaped nanoparticles, while DNA oligomers comprising poly T and poly G produced spherical nanoparticles. It was observed that poly G oligomers longer than 10 nucleotides had secondary structure due to internal folding, thereby forming a compact structure that is hydrophobic, and making poly G more difficult to use for nanoparticle synthesis. In an oligomer containing a mixture of nucleotides, as the percentage of A and C increases (such as a DNA oligomer containing at least 75% A and C nucleotides, at least 80%, at least 90%, or at least 95% A and C nucleotides, the flower morphology becomes more pronounced. However, if a large majority (e.g., at least 90%, at least 95%, at least 97% or at least 98%) of the nucleotides of the DNA oligomer are T, the nanoparticle will be spherical.

In some embodiments, it is beneficial to maximize the flower-like morphology of the nanoflower while minimizing the thickness of the deposited gold. Nanoflower growth can be monitored by the nanoparticle's UV absorbance. Gold spherical nanoparticles exhibit specific UV absorbance in the 500-600 nm range, and the absorption at this wavelength is a good indicator of the size and the polydispersity of the nanoparticles. As spherical nanoseeds grow into nanoflowers, the absorption peak will blue shift (increase in wavelength) and the absorbance at the original wavelength will decrease. By monitoring the subsequent shifted peak that corresponds to the formation of the nanoflower structure as well as the original peak of the nanosphere, it is possible to assign a quality factor to track the growth of nanoflowers that is expressed as:

Quality factor=Abs<Nanoflower>/Abs<Nanosphere>

The optimum gold concentration that maximizes nanoflower morphology with minimum gold growth can be determined by plotting this quality factor vs. the amount of gold salt added.

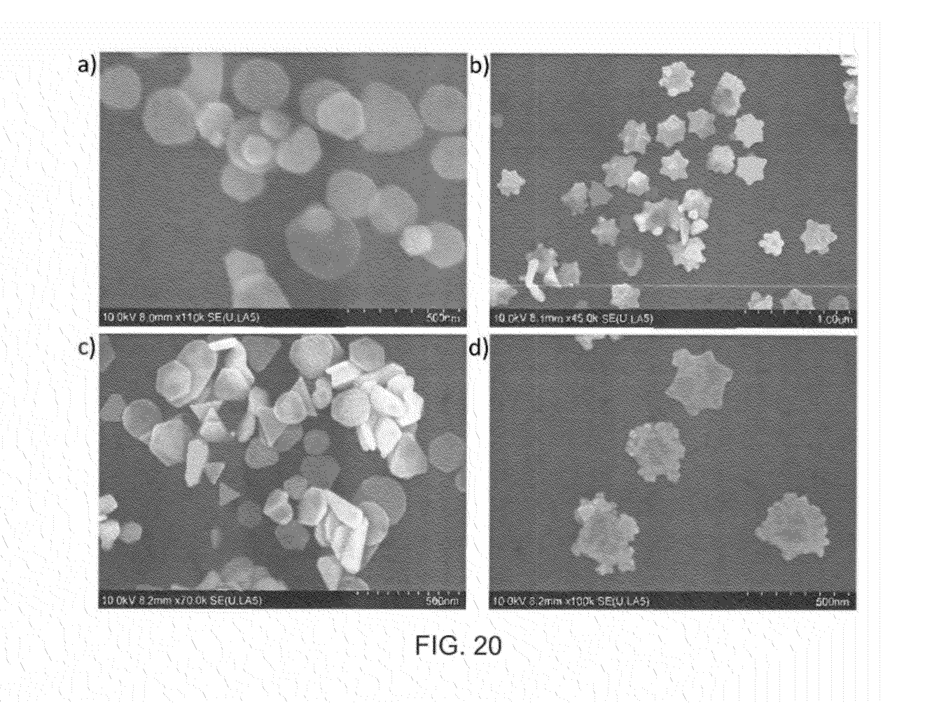

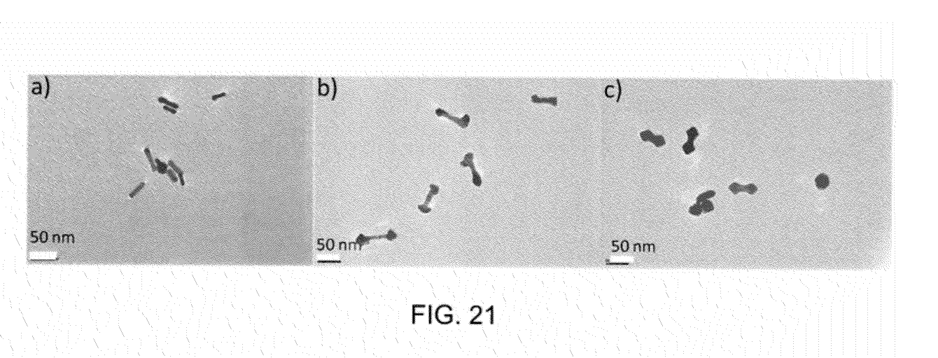

The sequence of the nucleic acid also mediates growth and morphology of nanoparticles synthesized from non-spherical seeds. In a working embodiment, when gold nanoprisms were functionalized with A30 or C30 DNA oligomers and additional gold was deposited, flat nanoplates were formed (FIGS. 20a, 20c). Thus, DNA oligomers of poly A or poly C, or a mixture of A and C (such as a DNA oligomer of at least 75% A and C), can be attached to gold nanoprisms to make flat nanoplates. In contrast, gold nanoprisms functionalized with T30 or G10 DNA oligomers formed multi-pointed nanostars (FIG. 20b, 20d). Thus, DNA oligomers of poly T or poly G, or a mixture of T and G (such as a DNA oligomer of at least 75% T and G), can be attached to gold nanoprisms to make multi-pointed nanostars. In another working embodiment, gold nanorods functionalized with A30 DNA oligomers produced bone-shaped, or dumbbell-shaped, nanoparticles (FIG. 21b), whereas nanorods functionalized with T30 oligomers produced nanoparticles resembling peanuts (FIG. 21c). Thus, DNA oligomers of poly A, or a mixture of A with other nucleotides (such as a DNA oligomer of at least 75% A), can be attached to gold nanorods to make dumbbell-shaped, nanoparticles, while DNA oligomers of poly T, or a mixture of T with other nucleotides (such as a DNA oligomer of at least 75% T), can be attached to gold nanorods to make nanoparticles resembling peanuts.

In certain embodiments, a nucleic acid sequence is selected based at least in part on its ability to bind to a target, e.g., a target protein. In such embodiments, it is desirable to control nanoflower size by selecting an appropriately sized nanoseed and then depositing a thin layer of gold so that only a minimal portion of the oligomer is buried in the deposited gold. For example, aptamer AS1411 (SEQ ID NO: 2) recognizes and binds to nucleolin, a eurkaryotic nucleolar phosphoprotein involved in the synthesis and maturation of ribosomes. In order to facilitate binding to its target, the entire AS1411 sequence preferably is fully exposed. Thus, in some embodiments, the nanoseed is functionalized with a plurality of oligomers comprising the aptamer plus an additional “tail” of nucleotides, e.g., a poly C tail, such that a portion of the tail is embedded in the deposited metal while the aptamer sequence remains fully exposed. Based on this teaching, one can select an appropriate aptamer based on the target, and incorporate the selected aptamer into the disclosed nanoparticles.

In some examples, the disclosed nanoparticles further include other molecules. In one example, the disclosed nanoparticles further include antibodies or fragments thereof that can be used to target a nanoparticle to a target cell. In one example, the antibody is specific for a cell surface receptor, such as a receptor on a cancer cell. Such nanoparticles can be used for example to image or treat (e.g., kill) the cancer cell. In another example, the disclosed nanoparticles further include a therapeutic molecule that can be used to treat a target cell. For example, the therapeutic molecule can be a drug that is used to treat a disease, such as a chemotherapeutic agent (e.g., cisplatin, doxorubicin, fluorouracil). In another example, the therapeutic molecule is a nucleic acid molecule used for gene therapy.

Chemotherapeutic agents are known in the art (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Exemplary chemotherapeutic agents that can be conjugated to a nanoparticle provided herein include but are not limited to, carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone and vinorelbine.

### III. NANOPARTICLE USES

Bio-functionalization of nanomaterials can provide the nanomaterials with target recognition ability, and can enable their controlled assembly.⁴¹ This functionalization step typically involves chemical modifications of the nanoparticles or the biomolecules to allow conjugation. For example, some embodiments of the disclosed shaped nanoparticles (e.g., nanoflowers, nanoplates, nanospheres, and/or nanostars), are capable of binding to and/or entering a target cell. In one embodiment, a nucleic-acid functionalized nanoparticle comprises an aptamer capable of binding to an antigen of interest. In another embodiment, a molecule of interest (e.g., an antibody, antibody fragment, peptide, protein, or drug molecule) is conjugated to a nucleic acid-functionalized nanoparticle. The molecule of interest may be conjugated to the nucleic acid oligomer extending from the nanoparticle, or the molecule of interest may be conjugated directly to the nanoparticle surface. Certain embodiments of the disclosed shaped nanoparticles are capable of forming larger nano-assemblies comprising a plurality of shaped nanoparticles. Additionally, some embodiments of the disclosed shaped nanoparticles have unique optical and/or electrical properties that may provide utility for imaging and/or biosensing applications, e.g., surface-enhanced Raman spectroscopy-based biosensing.

Nanoflowers have several advantages over nanospheres. For example, nanoflowers have a much higher surface area than nanospheres of a similar size. Therefore, more biomolecules or drug can be loaded on each nanoflower. In addition, the tips of the nanoscale protrusions and the nanocavities on the surface of gold nanoflowers have strong localized near-field enhancement effects, and they give a much stronger Raman signal enhancement effect than the gold nanospheres. Furthermore, preliminary studies indicate that AuNFs are more easily taken up and internalized by cells via endocytosis than non-functionalized gold nanospheres.

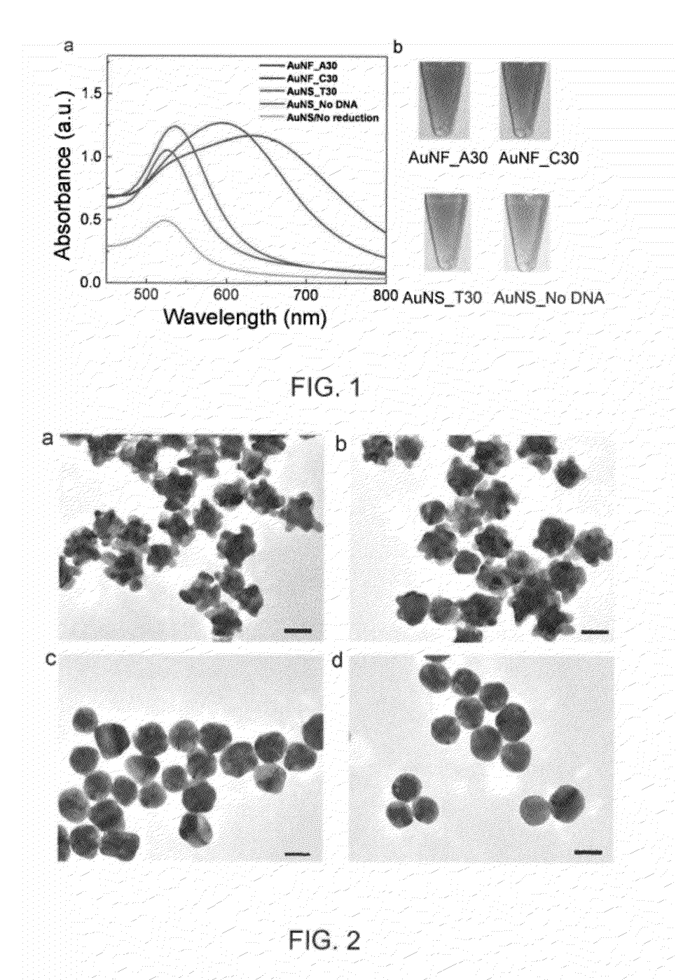

In some embodiments, nanoflowers also have different optical properties than nanospheres. For example, AuNFs have a peak absorbance at longer wavelengths (e.g., 600-630 nm) than gold nanospheres (unfunctionalized or functionalized with T30), which have a maximum absorbance at 520-530 nm (FIG. 1a). The absorbance shift allows visualization of AuNFs with near-infrared radiation, and also may make AuNFs suitable candidates for photothermal therapies since near-IR absorption increases the temperature of the AuNFs.

Nucleic acid-functionalized nanoflowers may be used as imaging agents and nano-carriers in a cellular environment. For example, some embodiments of DNA-functionalized AuNFs can be taken up by cells. Without being bound by any particular theory, it is believed that this cellular uptake ability might be due to high DNA loading on the AuNF surface and/or the morphology of the AuNF. Intracellular AuNFs scatter light and can be visualized using dark-field microscopy. The cellular uptake ability and light scattering property make the AuNFs promising nano-carriers for drug or gene delivery and promising contrast agents for intracellular imaging.

In certain embodiments, a nucleic-acid functionalized nanoflower comprises an aptamer capable of binding to an antigen of interest. DNA aptamers have been shown to be a useful targeting ligand for many biologically and medically relevant targets, and have shown potential for in vivo targeting applications.^{57,58} Thus, an aptamer-functionalized nanoflower can be used to deliver the nanoflower to a desired target (such as a particular cell type). In one embodiment, a gold nanoflower comprises an AS1411 aptamer, which binds specifically to nucleolin, a protein that is over expressed ˜20-fold on the surface of certain cancer cells and is an exemplary binding target for human breast cancer cells, e.g., MCF-7.

Molecules of interest (e.g., antibodies, peptides, proteins, drug molecules) may be attached to nucleic acid-functionalized gold nanoflowers by conventional coupling techniques. For example, molecules of interest can be attached to DNA-functionalized gold nanoflowers by conventional gold or DNA coupling techniques. In some embodiments, the nucleic acid oligomers may be chemically modified to facilitate functionalization with, e.g., antibodies, peptides, proteins, and/or drug molecules. In other embodiments, the molecules of interest may be attached directly to the nanoflower surface.

Nanoflower-antibody conjugates may be used to deliver NFs to desired targets. For example, an antibody that recognizes a particular target antigen on a cell surface may be conjugated to the NF. Alternatively, the NF may be conjugated to an antibody that recognizes, e.g., mouse monoclonal antibodies. In such an embodiment, a mouse monoclonal antibody specific for a target antigen may be administered to a subject where it binds to the target antigen, followed by administration of the anti-mouse antibody-NF conjugate.

In one embodiment, an antibody-NF conjugate may be used for imaging target cells. For example, antibodies to an antigen found on the surface of cancer cells may be conjugated to NFs. The antibody-NFs may be administered to a subject, with the antibody then recognizing and binding to the cancer cell antigens. The cancer cells may be imaged by any suitable method, such as CT or x-ray imaging.

In one embodiment, an antibody-NF conjugate may be used in photothermal and/or radiotherapy, e.g., for treatment of cancer. Photothermal therapy is a technique that converts electromagnetic radiation (usually in the form of infrared) into thermal energy as a therapeutic technique for medical conditions, such as cancer. Gold and silver nanoparticles have emerged as powerful platforms for in vitro and in vivo biomedical applications, due to their high stability, low toxicity, and ability to be taken up by cells.⁵⁹ As the dimensions decrease in metals, the properties of the surface become dominant and give nanoparticles new properties. As the dimensions decrease in metals, the properties of the surface become dominant and give nanoparticles new properties. In noble metals, the coherent collective oscillation of electrons in the conduction band induces large surface electric fields which greatly enhance the radiative properties of gold and silver nanoparticles when they interact with resonant electromagnetic radiation. This makes the absorption cross section of these nanoparticles orders of magnitude stronger than that of the most strongly absorbing molecules and the light scattering cross section orders of magnitude more intense than that of organic dyes. It was realized that this intense absorption provided a path to efficiently convert IR light to an intense local heating around the nanoparticle.⁶⁰ Photothermal therapy places these metal nanoparticles only in and around diseased and/or cancerous cells to create localized heating that would selectively kill the targeted cells without damaging the surrounding area.⁶¹

In order to be considered applicable for in vivo applications, nanoparticles should absorb EM radiation most efficiently from 700 nm to 900 nm, also known as the near IR window where skin, tissues, and hemoglobin have minimum absorption and scattering, allowing the radiation to penetrate deep into the tissue. The efficiency with which a nanoparticle can convert near IR radiation to thermal energy is partly determined by electric fields that arise from the oscillations of surface electrons. Sharp, pointed features, such as the morphological features of nanoflowers, behave as focusing points for such oscillations and can dramatically increase the radiative properties at these locations.

Gold nanoflowers have been shown to absorb energy in the near-infrared region. Absorption of NIR energy will increase the temperature of the AuNF. Thus, an antibody-AuNF conjugate bound to a cancer cell may be irradiated with NIR radiation, thereby heating the AuNF and destroying the cancer cell. Alternatively, the AuNFs may be used to increase the dose of x-ray radiation received by the cancer cells relative to the dose received by normal tissue. The absorption characteristics of AuNFs may allow effective treatment (e.g., cancer cell destruction) with less radiation than conventional gold nanospheres.

In other embodiments, nanoflowers may be used to deliver molecules of interest to a cell. For example, a drug molecule may be conjugated to the NF surface or to the nucleic oligomers protruding from the NF surface. Because cells can take up DNA-functionalized AuNFs (see Example 6), the AuNF may be used to deliver a drug molecule to the cell interior. Alternatively, coupling a drug molecule to an NF-antibody conjugate may be used to deliver the drug to the immediate environment, or vicinity, of a targeted cell. Thus, an anti-cancer drug, for example, could be delivered specifically to a tumor site rather than disseminated throughout the body. Such methods can be used in combination with other therapies, such as other anti-neoplastic therapies, such as radiation therapy, chemotherapy immunosuppressants (such as Rituximab, steroids), and cytokines (such as GM-CSF).

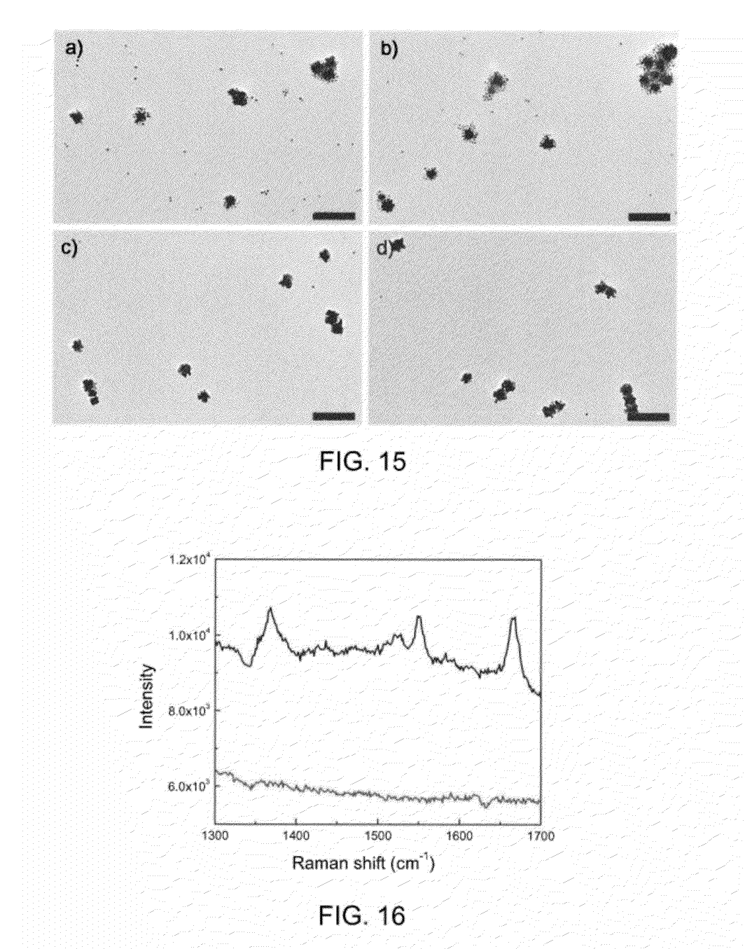

Nanoparticles prepared by embodiments of the disclosed method also can be used to make nano-assemblies. Nucleic acid-directed nano-assemblies may be used for biosensing and nanoscale photonic device applications. A nanoflower functionalized with oligomers of a given sequence can be prepared. The oligomers can act as ligands to bind and attach additional nanoparticles to which complementary oligomers are attached. For example, nanoparticles functionalized with poly T oligomers can bind to a gold nanoflower functionalized with poly A oligomers via the interaction between the poly A and poly T oligomers. (See, e.g., FIG. 14a.) However, if the added nanoparticles include non-complementary oligomers, then little or no binding occurs. For example, nanoparticles with bound poly A oligomers will not bind to a poly A-nanoflower. Thus, formation of the nano-assemblies is sequence specific. Additionally, the number of oligomers on the “central” nanoparticle, or nanoflower, determines in part how many “peripheral” nanoparticles including complementary oligomers can be attached to form the nano-assembly. As the number of oligomers on the central nanoparticle increases, so does the number of peripheral nanoparticles that can assemble onto it. One of ordinary skill in the art will understand that the number of nanoparticles in the nano-assembly also depends at least in part upon space constraints and the relative sizes of the nanoparticles. A larger central nanoparticle can accommodate more peripheral nanoparticles than a smaller central nanoparticle. Similarly, using smaller peripheral nanoparticles allows more nanoparticles to assemble onto the central nanoparticle.

These flower-like nanoparticles may also have promising applications in SERS (Surface Enhanced Raman Spectroscopy) based biosensing. Raman spectroscopy is a useful technique that detects and identifies molecules based on their vibrational energy levels and corresponding Raman fingerprints. However, Raman scattering from the molecules themselves without enhancement is very weak. Colloidal Au nanospheres have been used to increase the scattering efficiencies of Raman-active molecules by as much as 10¹⁴-10¹⁵-fold.⁴⁴ Compared to these AuNSs with smooth surfaces, AuNFs may be a better candidate for fabricating SERS-active tags for a number of reasons: (i) the tips of the nanoscale bumps and the nanocavities on the AuNF surface have strong localized near-field enhancement effects^{45,46}; (ii) AuNFs have a larger total surface area due to the roughness of the AuNF surface; and/or (iii) the surface plasmon resonance peaks of the AuNFs (e.g., 630 nm for AuNFs) are nearer to the excitation wavelength, which provides stronger enhancement effects.

Nanoparticles with different shapes have different physiochemical properties. Thus, new nanoparticle shapes such as nanoplates, nanostars, etc., have unique optical and/or electrical properties that are significantly different from nanospheres or nanoflowers. These new nanoparticles may have an improved performance in SERS sensing, and imaging and drug delivery in comparison with nanospheres. Furthermore, these nanoparticles with different light scattering properties may also be used collectively for multiplex sensing or imaging by encoding each target with a different type of nanomaterial. For example, a nanoplate may be functionalized (e.g., with an antibody or an oligonucleotide probe) to couple to one target, while a nanostar may be functionalized to couple to a different target.

### IV. EXAMPLES

### Chemicals and Materials

All oligonucleotides used herein were purchased from Integrated DNA Technologies Inc. (Coralville, Iowa). Solutions of 20-nm and 5-nm gold nanospheres (AuNSs) were purchased from Ted Pella (Redding, Calif.) and purified using a centrifuge before use. Hydrogen tetrachloroaurate(III) hydrate (HAuCl₄.3H₂O, 99.999%; Sigma-Aldrich), hydroxylamine hydrochloride (NH₂OH.HCl, 99.9999%; Sigma-Aldrich), sodium hydroxide (NaOH, 98%; Sigma-Aldrich), adenosine 5′-monophosphate sodium salt (AMP, 99%; Sigma-Aldrich), tris(2-carboxyethyl)phosphine hydrochloride (TCEP, C₉H₁₅O₆P.HCl; Sigma-Aldrich), 2-mercaptoethanol (ME, 98%; Sigma-Aldrich) and mPEG thiol (CH₂O—(CH₂CH₂O)₆—CH₂CH₂SH, Mw=356.5; Polypure) were used without further purification.

### Characterization Methods

Shapes and sizes of gold nanoparticles as well as the nano-assemblies were analyzed using a JEOL 2010LaB6 transmission electron microscope (TEM) operated at 200 kV. Samples were prepared by putting a drop of a nanoparticle solution onto a carbon-coated copper TEM grid (Ted pella).

Absorbance of the nanoparticle solutions was characterized using UV-Vis spectrophotometry (Hewlett-Packard 8453).

Darkfield light-scattering images were acquired using a Zeiss Axiovert 200M inverted microscope coupled with a CCD digital camera. The individual nanoparticles on a glass coverslip were imaged using an EC Epiplan 50× HD objective (NA=0.7), and the Chinese hamster ovary (CHO) cells were imaged with a Plan-Neofluar 10× objective (NA=0.3). Prior to acquisition, the digital camera was white-balanced using Zeiss Axiovision software so that colors observed in the digital images represented the true color of the scattered light.

Z-stacks of fluorescence images of the cells were acquired using Andor Technology Revolution System Spinning Disk Confocal Microscope at 100× objective (oil immersion, excitation wavelength 488 nm). The collected z-stacks of images were then deconvoluted and assembled into a 3D image using Autoquant X software and Imaris software.

### Example 1

### Nanoparticle Synthesis and Characterization

The concentration of purified 20-nm citrate-coated gold nanospheres (AuNSs) was calculated based on the Beer-Lambert law (extinction coefficient of 20-nm AuNS at 520 nm is 9.406*10⁸ M⁻¹ cm⁻¹) and then adjusted to 0.5 nM and resuspended in pure water. A 300 μL aliquot of 0.5 nM 20-nm AuNS solution was first incubated with 1 μM of DNA (poly A30, poly C30 or poly T30) for 15 min to let DNA adsorb onto the AuNS surface. This step was followed by addition of 15 μL of 400 mM NH₂OH (adjusted to pH 5 with NaOH) to produce a final concentration of 20 mM NH₂OH. Three types of 30-mer DNAs consisting of poly A, poly C, or poly T (designated as A30, C30, and T30, respectively) were used. After vortexing, 2.1 μL 1% (wt/wt) HAuCl₄ was introduced to AuNS mixture solution (final concentration of HAuCl₄ was 167 μM), and the mixture was rigorously vortexed to facilitate the reduction. A color change was observed in seconds. The mixture solution was constantly vortexed for another 15 min until the reaction was complete. Based on the DNA sequences used and their shape, the synthesized gold nanoparticles were called AuNF_A30, AuNF_C30 or AuNS_T30 respectively. Surprisingly, nanoparticle solutions synthesized in the presence of A30 or C30 were blue colored, while the nanoparticle solution synthesized with T30 was red colored (FIG. 1b). The resultant solutions were stable for days without showing any nanoparticle aggregation or color change.

To determine the morphology of the nanoparticles prepared with different DNA sequences, transmission electron microscopy (TEM) was employed to investigate each of the resulted nanoparticle solutions. Surprisingly, those particles synthesized with A30 or C30 were flower shaped (designated as AuNF_A30 and AuNF_C30) (FIGS. 2a, 2b), while particles synthesized with T30 were spherical (AuNP_T30, FIG. 2c). The flower-shaped gold nanoparticles had a broad surface plasmon absorbance that peaked at 600 nm (for AuNF_C30) or 630 nm (for AuNF_A30) (FIG. 1a), which is consistent with the absorbance of gold nanoflowers prepared by other reported methods.³⁶

Poly G30 was not tested due to synthetic difficulties caused by the formation of a guanine tetraplex structure.³⁷ Instead, a shorter DNA consisting of 10-mer poly G was tested, and the resulting nanoparticles were nearly spherical (FIG. 3). In contrast, only spherical nanoparticles were formed in the absence of DNA (FIG. 2d) or in the presence of salt only (FIGS. 4a and b).

No metal nanoparticles were formed upon mixing DNA, NH₂OH and HAuCl₄ together, without the addition of AuNS as seeds. These results demonstrated that the DNA mediates the morphology of the gold nanoparticles, and the nanoparticle shape is sequence dependent.

To understand the DNA sequence-dependent nanoparticle formation and to determine the stability of DNA-adsorbed AuNs, the adsorption step of single-stranded DNA (ssDNA) on AuNS was investigated. Unmodified ssDNA is able to adsorb onto AuNS, and enhances the electrostatic repulsion between AuNSs, thereby reducing or preventing salt-induced aggregation.³⁸ First, 100 μL of 1 nM, 20 nm AuNS solutions were incubated with 1 μM DNA (either poly A30, poly C30, or poly T30, respectively). After 15 min incubation, 0.1 M NaCl was introduced to each of the solutions. UV-vis spectroscopy was used to record the absorbance of each solution before and after the addition of NaCl.

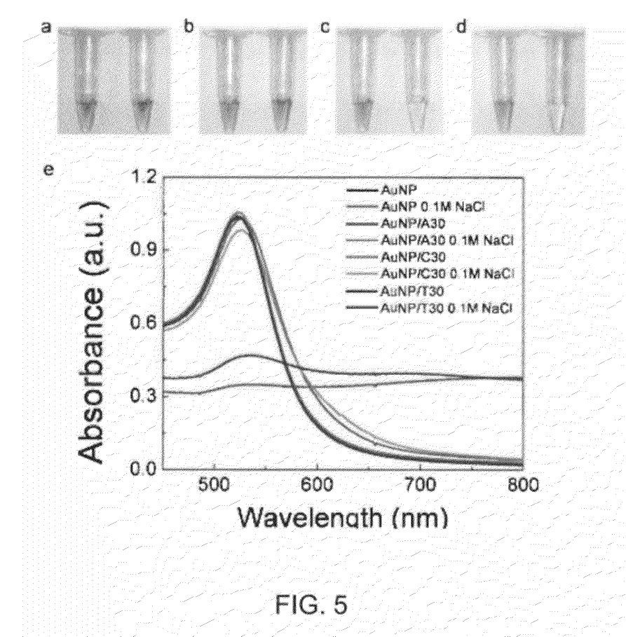

As shown in FIGS. 5a-e, aggregation of AuNS happened immediately when the T30 DNA sequence was used for incubation with the AuNS, while AuNS incubated with A30 or C30 sequences remained stable. Since the stability of the AuNS at the same salt concentration is determined by the number of DNA adsorbed on its surface,39 it was concluded that many fewer T30 molecules were adsorbed onto the AuNS surface compared to A30 or C30, which is consistent with the lower binding affinity of T30 towards the gold nanoparticle surface. This result explains the differences in shaping the gold nanoparticle by the T30 sequence in comparison with A30 or C30.

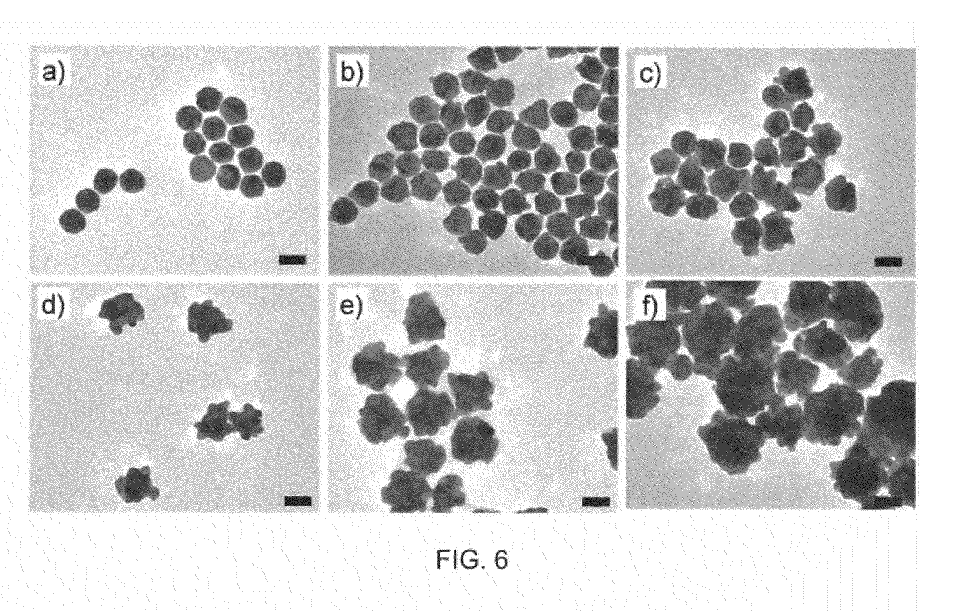

To further evaluate the mechanism of shape control process of the flower-shaped nanoparticle directed by DNA, varying amounts of HAuCl₄ were added to A30, which was incubated with AuNS and 20 mM NH₂OH to initiate the reduction. Since NH₂OH was in large excess, it was expected that the HAuCl₄ would be completely reduced to gold metal in the presence of AuNS seeds.³⁵ As shown in FIGS. 6a-f, with the addition of increasing amount of HAuCl₄, the resultant nanoparticle evolved from sphere shape to a bud sphere and then into the flower-like shape. Upon further increase of the HAuCl4 amount, the flower shaped nanoparticle would grow even bigger.

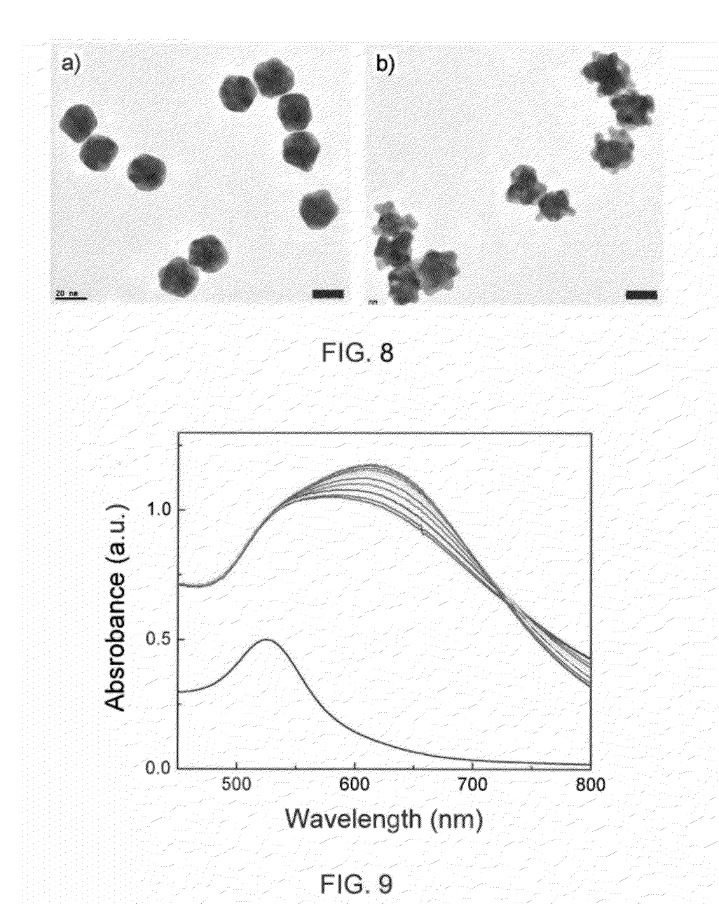

In order to investigate how the nanoparticle morphology was affected by the number of DNA oligomers adsorbed on AuNS, varying amounts of A30 were incubated with AuNS and followed by reduction of equal amounts of HAuCl₄. FIGS. 7a-f shows that the nanoparticle shape changed from spherical to flower-like with increasing numbers of DNA oligomers adsorbed on AuNS, while the size of the gold nanoparticle remained the same. From the above observations, it was determined that DNA of chain-like structure was able to direct the deposition of the reduced gold metal on the AuNS and guide the nanoparticle growth from a spherical into a flower-like shape. This conclusion was further supported by the control experiments, which showed that when the single deoxynucleotide, adenosine monophosphate (AMP) was incubated with AuNS instead of a DNA chain, the nanoparticles obtained were nearly spherical, while a random 30-mer DNA sequence of mixed A, T, G, C caused the formation of flower-shaped nanoparticles (FIGS. 8a-b).

To further probe this DNA mediated AuNF growing process, the absorbance of AuNF growth solution was monitored using UV-visible spectroscopy. As shown in FIG. 9, after initiation of the reaction for 3 seconds, the intensity of the nanoparticle absorbance increased significantly, and the peak of the AuNSs at 520 nm broadened and red-shifted. With growth of the AuNS, a new absorbance peak at 630 nm from the resultant AuNFs appeared, and the reaction completed in about 15 minutes.

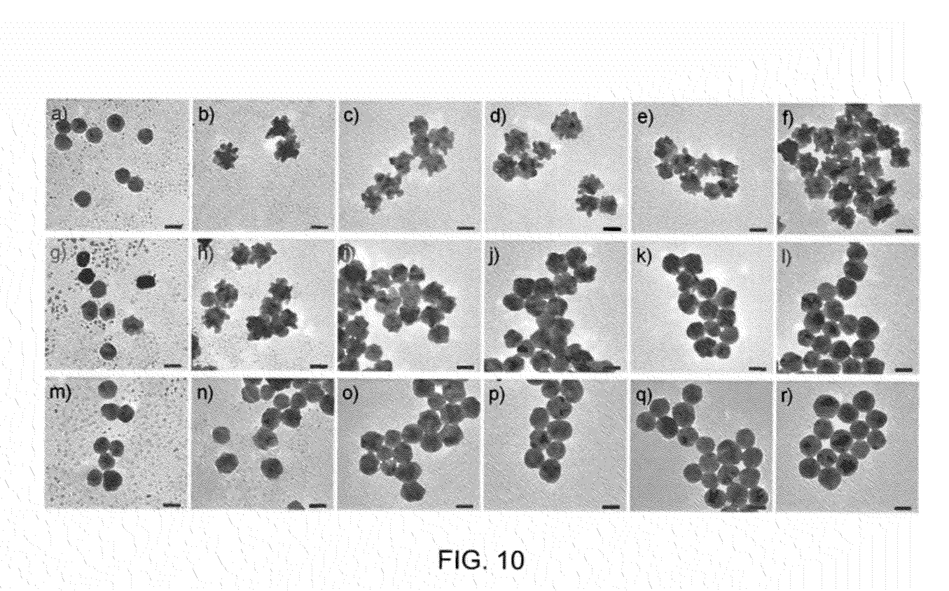

This time-dependent AuNF growth process was further studied using TEM by stopping the reaction at the early stages of NP growth with excess mercaptopropionic acid (MPA). MPA has been shown to quench the NP growth effectively by forming the less reactive Au(I)-MPA complex with gold ion.⁴⁰ As shown in FIGS. 10a-r, both the 20-nm AuNSs and 1-3 nm small nanoparticles (SNPs) could be observed after initiation of the reaction at 0.5 second.

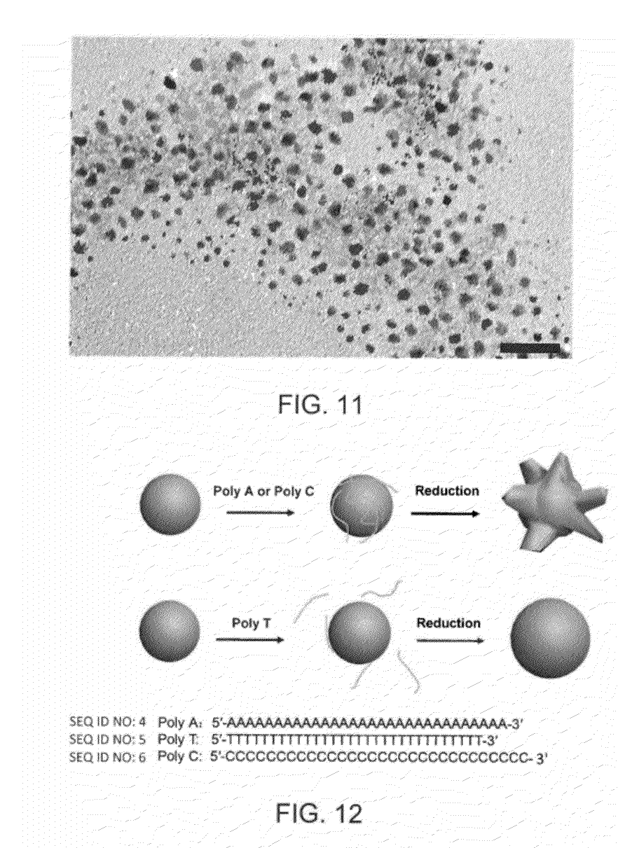

A further control experiment showed that formation of the SNPs could be due to the conversion of Au(I)-MPA complexes into metal particles on the TEM grid upon electron-beam irradiation during TEM imaging (FIG. 11). Flower-like nanoparticle intermediates were observed after 2 seconds of reaction in both A30 and T30 mediated syntheses. Interestingly, the flower-like intermediates prepared with T30 grew further into nanospheres within 30 s while the intermediates prepared with A30 maintained their flower-like structure and stable AuNFs were produced. In the absence of DNA, the AuNSs grew into bigger nanospheres and no flower-like intermediate was observed. These results suggest that DNA adsorbed on the AuNS surface acts as a template to mediate the formation of flower-like gold nanoparticles. The formation of the AuNF results from either selective deposition of the reduced gold metal on AuNS templated by surface-bound DNA or from uneven growth of the AuNS due to the binding of DNA to the surface.

As depicted in FIG. 12, due to the strong binding affinity of poly A (SEQ ID NO: 4) or poly C (SEQ ID NO: 6) to AuNS, a number of A30 or C30 bind tightly to AuNS and induce the inhomogeneous growth of AuNS, producing the flower-like nanoparticles. In contrast, fewer poly T molecules bind weakly and loosely to AuNS. The weakly bound poly T molecules produce the flower-like intermediates at a very initial stage. However, they are not able to stabilize the flower-like structures, and the spherical particles are eventually formed.

### Example 2

### Determination of the Number and Stability of Thiolated and Unmodified Oligonucleotides on Gold Nanoflowers

### Preparation of Thiolated DNA-Gold Nanoflowers

Functionalization of thiolated DNA (HS-A30 or HS-T30) on 5-nm gold nanospheres was carried out by following a published protocol⁵⁵ with slight alterations. Briefly, 9 μL of 1 mM thiolated DNA was first mixed with 1.5 μL of 10 mM TCEP (tris(2-carboxyethyl)phosphine) solution and 1 μL of 500 mM acetate buffer (pH 5.2) to activate the thiolated DNA. After a 30-minute reaction, the mixture was transferred into 3 mL of 5-nm AuNS solution (82 nM, in pure water) followed by addition of 10 mM Tris-HCl buffer (Tris=2-amino-2-hydroxymethyl-1,3-propanediol, pH 8.2). The nanoparticle solution was incubated overnight, and the NaCl concentration was then increased to 0.1 M. The functionalized 5-nm AuNS solutions (designated as AuNS₅ₙₘ_{_{—}}S_A30 or AuNS₅ₙₘ_{_{—}}S_T30) were incubated for another 12 h before usage. To purify the nanospheres from the unreacted DNA, a Microcon® centrifugal filter (Ultracel YM-100, MWCO=100K; Millipore, Billerica, Mass.) was used by following the instructions from the manufacturer.

### Preparation of Unmodified DNA Gold Nanoflowers

Fluorophore (FAM) labeled poly A30 was used for AuNF synthesis. The AuNFs were synthesized by incubating 1 μM of Fluorophore (FAM) labeled poly A30 (FAM-A30) with 300 μL of 0.5 nM 20 nm AuNS solution for 15 min. 15 μL of 400 mM NH2OH (pH 5) and 2.1 μL 1% (wt/wt) HAuCl4 were added to the nanoparticle solution to initiate the AuNF formation (three samples were prepared separately). Meanwhile, 300 μL 1 μM FAM-A30 solutions were prepared with the addition of 15 μL of 400 mM NH₂OH (pH 5) and 2.1 μL pure water and these solutions were used as control solutions. After AuNF synthesis, the supernatants were collected by removing the nanoparticles with centrifugation. The oligonucleotide concentrations in both the collected supernatants and the control solutions were quantified and compared by using UV absorbances at 260 nm. The DNA concentration in the supernatants was 825.6 nM, so the DNA attached to the AuNFs during synthesis were 174.4 nM. Dividing this number by the AuNS concentration (0.5 nM), it was estimated that the average number of attached oligonucleotides on each AuNF was ˜349.

### Stability of Attached Oligonucleotides.

To probe the stability of the DNA attached to AuNFs, the number of oligonucleotides on AuNFs after treatment with mercaptoethanol was quantified using a fluorescence-based method.⁵⁶ The AuNF solutions (0.5 nM) were treated with mercaptoethanol (ME) to a final concentration of 14 mM overnight. The solutions containing the displaced oligonucleotides were separated from AuNFs by centrifugation. Each supernatant (100 μL) was added to 400 μL 62.5 mM phosphate buffer (pH 7.2). The pH and ionic strength of the sample and calibration standard solutions were kept the same for all measurements due to the sensitivity of the fluorescent properties of FAM to these conditions. The fluorescence maximums (520 nm) were measured and then converted to molar concentrations of the FAM labeled oligonucleotides by using a standard linear calibration curve. Standard curves were carried out with known concentrations of fluorophore-labeled oligonucleotides under same buffer pH, salt, and mercaptoethanol concentrations.

The average number of displaced oligonucleotides for each AuNF was obtained by dividing the calculated oligonucleotide molar concentration by the original AuNF concentration. The results demonstrated only ˜110 strands were displaced by mercaptoethanol (ME), and the majority (˜240 strands) was still bound to the AuNF after the treatment. Thiol-gold chemistry is the most used method to conjugate DNA to gold surface. Under the same ME (14 mM) treatment, however, all of the thiolated DNA oligonucleotides were displaced by ME from the gold surface.⁴²

### Example 3

### Melting Point Determination of DNA-Functionalized Gold Nanoflowers

Considering the remarkably high binding affinity of DNA to the AuNFs (higher than thiol-gold binding), it was hypothesized that the DNA in situ attached to AuNFs during reduction could be partially buried in the AuNFs. To test this hypothesis and also the functionality of the DNA on the AuNFs, experiments were performed to test the melting point of the DNA in-situ attached on the AuNFs.

AuNFs were first treated with thiolated PEG (polyethylene glycol, 6 μM) molecules overnight to displace any weakly bound DNA on AuNF surfaces.⁴³ Purified AuNF_A30 (2 nM) was hybridized with fluorophore (FAM) labeled Poly T30 (FAM-T30) (1 μM) in a buffer solution containing 10 mM HEPES buffer (pH 7.1) and 50 mM NaCl. The mixture solution was heated up to 65° C. and cooled down to room temperature in about two hours. The unhybridized fluorophore strands were removed by centrifugation, and the AuNFs (2 nM) were redispersed in the same buffer solution.

A fluorimeter (FluoroMax-P; Horiba Jobin Yvon, Edison, N.J.) coupled with a temperature controller was used to obtain the melting curve of the DNA hybridization on AuNFs. Since a gold nanoparticle can effectively quench the fluorescence from its surrounding fluorophores, the release of the fluorophore labeled DNA from AuNFs due to DNA melting will result in a fluorescence increase of the nanoparticle solution. The sample was kept at target temperatures for 72 seconds after the temperature was reached to ensure that the sample was at the stated temperature during data collection at each temperature. As a comparison, free A30 labeled with an organic quencher (Blank Hole Quencher-1, 200 nM)) was hybridized with FAM-T30 (200 nM) in the same buffer under identical conditions, and its melting curve was collected as well.

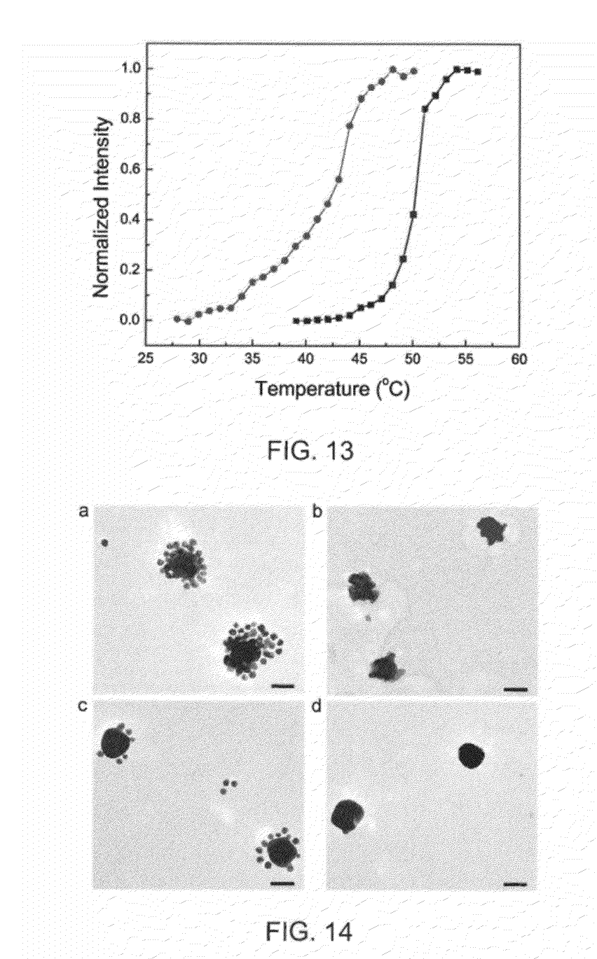

As shown in FIG. 13, the melting temperature of the DNA in situ attached to AuNFs (around 42° C.) was significantly lower than the free DNA (around 50° C.). This result indicated that a small segment of DNA might be buried in the AuNFs during the nanoparticle growth, while the majority part of DNA exposed outside was still functional for DNA hybridization.

### Example 4

### Nano-Assembly of DNA-Functionalized Gold Nanoparticles

The synthesized AuNF_A30 solution was first purified by centrifugation (9000×g, 5 min.) twice and then redispersed in water. The AuNF_A30 particles were then treated with 6 μM mPEG thiol for 2 hours and purified. After purification, AuNF_A30 (0.5 nM) was mixed with purified AuNS (50 nM) modified with thiolated complementary DNA (AuNS₅ₙₘ_{_{—}}S_A30 or AuNS₅ₙₘ_{_{—}}S_T30 respectively) in the presence of 10 mM phosphate buffer (pH 8) and 0.1 M NaCl. The mixture solution was incubated overnight to allow nano-assembly. The same procedure was used to assemble AuNS_T30 with AuNS₅ₙₘ_{_{—}}S_A30 or AuNS₅ₙₘ_{_{—}}S_T30. After incubation, the nanoparticle mixture solution was centrifuged at (9000×g, 2 min.) to remove free 5-nm gold nanoparticles in the supernatant, and the pellet was redispersed in buffer solution for TEM sample preparation.

TEM was then employed to assess the assembly of the nanoparticles. As shown in FIG. 14a, AuNF_A30 was surrounded by a number of AuNS5nm_S_T30, forming the satellite structure. As a comparison, when 5-nm AuNS functionalized with non-complementary DNA A30 (AuNS5nm_S_A30) were used to incubate with AuNF_A30, no assembly was observed (FIG. 14b). Additional large-area TEM images containing multiple satellite assembled nanostructures are shown in FIGS. 15a-d. These results further confirmed that the DNA molecules were not only densely functionalized to AuNFs in a large number, but also retained their molecule recognition properties. Interestingly, when AuNS_T30 were incubated with AuNS5nm_S_A30 under similar conditions, only a few 5-nm particles were assembled on AuNS_T30, while little assembly was observed with non-complementary AuNS5nm_S_T30 (FIGS. 14c, 14d). This observation indicates that fewer numbers of T30 oligonucleotides were attached during synthesis, consistent with the fact that fewer T30 oligonucleotides were adsorbed on AuNS compared to A30 or C30.

### Example 5

### Surface Enhanced Raman Spectroscopy of DNA-Functionalized Gold Nanoflowers

SERS enhancement from DNA functionalized AuNFs was compared with AuNSs. Raman tag labeled DNA (Trama-A30) was used to grow AuNFs and then the Raman signal was collected. As shown in FIG. 16, under the same conditions (excitation (603 nm), nanoparticle concentration (0.5 nM), etc.), the Raman signal from the Raman tag with the AuNFs was clearly observed while the signal from the Raman tag with AuNS was too low to distinguish. These results indicated that AuNFs provide a much stronger SERS effect over the AuNS. The Raman spectrometer was a home-made instrument located at Materials Research Lab at University of Illinois.

### Example 6

### Cellular Uptake of Gold Nanoflowers

AuNFs were synthesized with 1 μM of fluorophore (FAM) labeled poly A30 (FAM-A30) by following the procedure in Example 2. The AuNFs were purified by centrifugation.

CHO (Chinese hamster ovary) cells were cultured in Dulbecco's modified eagle medium (DMEM; Cell Media Facility, University of Illinois at Urbana-Champaign, Urbana, Ill.) supplemented with 10% fetal bovine serum (FBS), penicillin (50 U/ml), and streptomycin (50 μg/ml), at 37° C. in a humidified atmosphere of 5% CO2. Cells were seeded at a density of 1×10⁵ cells/cm² on 4 well Lab-Tek chambered #1 Borosilicate coverglass system (Fisher Scientific), and the cells were grown for 24 hours before treatment with nanoparticles. After 18 hours, the cells were washed with 1×PBS buffer and fresh media was added.

To investigate the cell uptake of the AuNFs, nanoparticles (0.5 nM or 1 nM) synthesized with fluorophore (FAM) labeled A30 were added to the cells and incubated for 18 hours. Excess AuNFs were removed by washing the cells with 1×PBS five times prior to imaging.

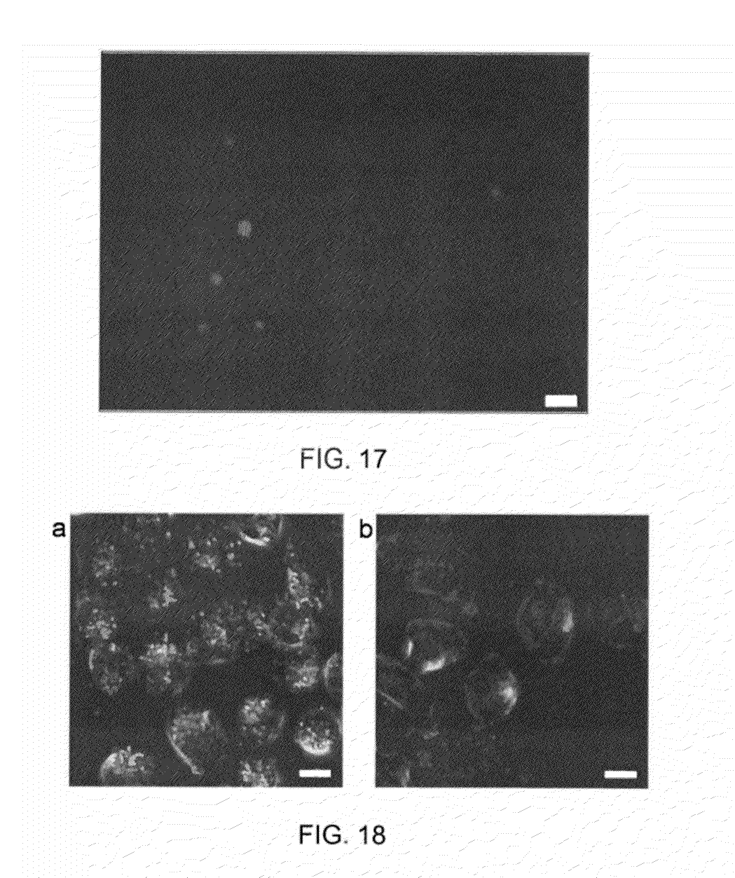

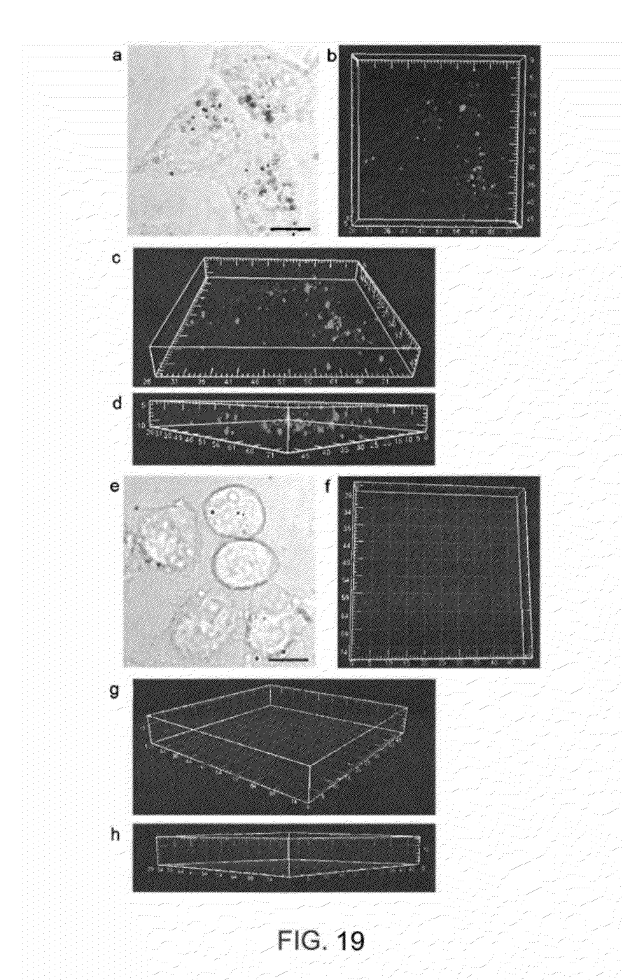

Dark-field light-scattering images were taken to visualize the AuNF uptake by the cells.⁴⁷ The light scattering property of the AuNFs was first investigated using a dark-field microscope coupled to a CCD digital camera. The digital camera was white-balanced so that the observed colors represented the true color of the scattered light. The AuNFs showed bright orange color in the dark field image (FIG. 17). As shown in FIG. 18a, the orange dots representing the AuNFs were observed in the intracellular region of the cells while the untreated control cells appeared dim yellow to green color due to the intrinsic cellular scattering (FIG. 18b). This nanoparticle cellular uptake was further confirmed by the 3-D reconstructed confocal microscope images of the AuNF treated cells, showing that the AuNFs were distributed inside the cells (FIGS. 19a-h). The results demonstrated that AuNFs entered into cells during the incubation. It is believed that this ability of the AuNF to be taken up by the cell might be due to the high DNA loading on the AuNF surface⁴⁸ and/or the shape effect.⁴⁹ The cellular uptake ability and light scattering property make the AuNFs promising nanocarriers for drug or gene delivery and contrast agents for intracellular imaging.

### Example 7

### Synthesis of Non-Spherical Nanoparticles

Gold nanoprisms were synthesized in the presence of surfactants and iodine by following a previously reported method.⁵⁰ After removing the free surfactant with centrifugation, these purified nanoseeds were incubated with DNA of different sequences (A30, T30, C30) respectively for 15 minutes. NH₂OH and HAuCl₄ were then added to the nanoparticle solution to initiate the particle growth.

The morphologies of the prepared nanoparticles were studied using scanning electron microscopy (SEM). Surprisingly, the nanoprisms incubated with A30 or C30 grew into thicker round nanoplates, while nanoprisms incubated with T30 grew into 2-D six-angled nanostars (FIGS. 20a-c). Nanoprisms incubated with G10 also produced 2-D multiple angled nanostars were produced (FIG. 20d). These results demonstrated that DNA of different sequences could direct the growth of the nanoprism into different shapes, and each sequence encodes the formation of nanoparticles with certain shapes.

Nanoparticle growth was also tested using gold nanorods as seeds. Remarkably, the nanorods (FIG. 21a) were converted into dogbone-like nanoparticles in the presence of A30 after growth (FIG. 21b), while the nanorods were converted into peanut-like nanoparticles in the presence of T30 (FIG. 21c).

These results indicate that embodiments of the DNA-mediated shape-control method can be readily adapted to synthesize other non-spherical nanoparticles. This method can be used as a general methodology to control growth of metal nanoparticles, and holds great promise to produce a series of novel nanoparticles with different shapes and unique properties.

### Example 8

### Nanoflower Size and Quality Control

Nanoflower size can be precisely controlled by controlling the growth conditions for nanoflowers, e.g., by varying the amount of gold available and/or by varying the nanoseed size.

In one example, nanoflowers were synthesized using 300 μL of a 0.5 nM solution of 13-nm gold nanoseeds (synthesized according to available protocols) with increasing amounts of a 1% w/v solution of HAuCl₄, and the resulting nanoflowers were analyzed by TEM. The nanoseeds were incubated with an AS1411 aptamer (1 μM; SEQ ID NO: 2) or a randomized control construct (1 μM; SEQ ID NO: 1) prior to gold salt reduction. The protocol described above in Example 1 was followed during synthesis.

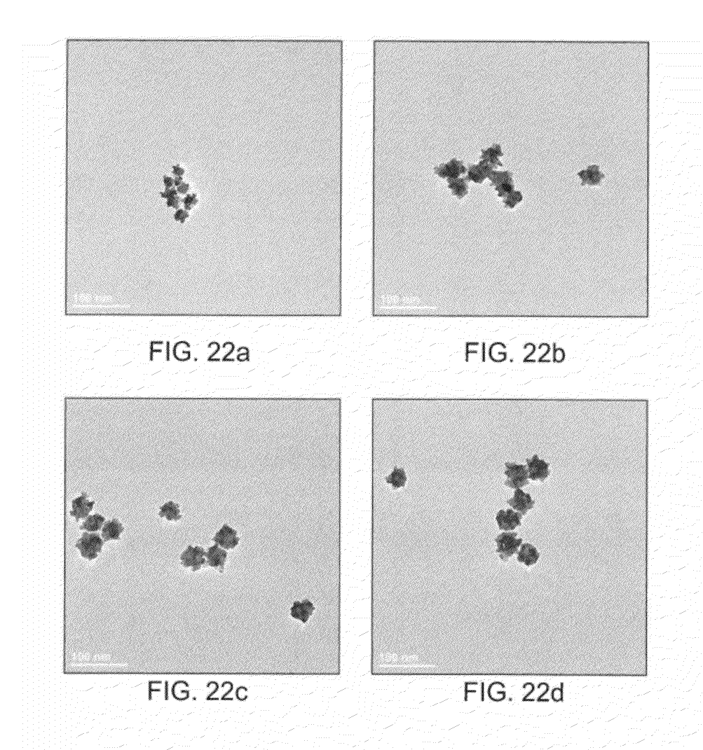

As shown in FIGS. 22a-d, increasing gold salt concentration under identical conditions leads to increasing nanoflower size with good uniformity. Using additional gold resulted in non-uniform structures (not shown). FIGS. 22a-d are TEM images of nanoflowers synthesized with the AS1411 aptamer under the following conditions:

TABLE 1

| | 0.5 nM, 13 nm seed | NH₂OH (400 mM) | 1% w/v HAuCl₄ |
|---|---|---|---|
| FIG. 22a | 300 μL | 15 μL | 0.7 μL |
| FIG. 22b | 300 μL | 15 μL | 0.9 μL |
| FIG. 22c | 300 μL | 15 μL | 1.3 μL |
| FIG. 22d | 300 μL | 15 μL | 1.5 μL |

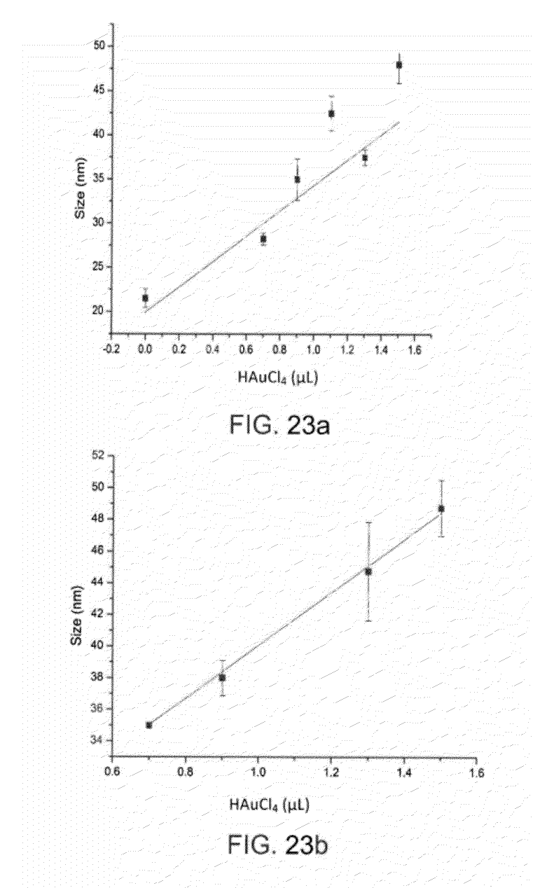

The relationship between gold salt concentration and nanoflower size was determined to be linear (FIGS. 23a-b). The nanoflowers in FIG. 23a were synthesized with the randomized DNA construct, and the nanoflowers in FIG. 23b were synthesized with the AS1411 aptamer.

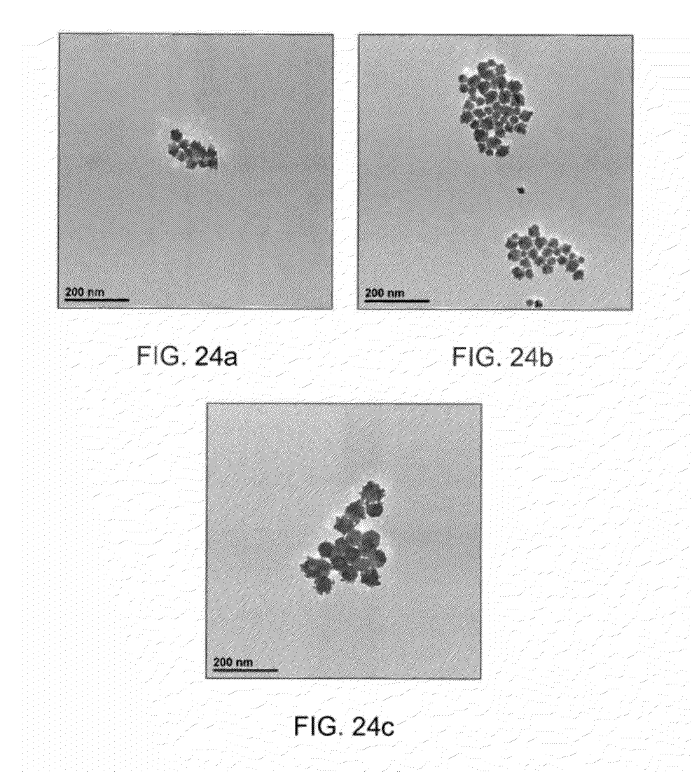

In another example, nanoflower size was controlled by varying the size of the nanoseed. Nanoflowers were synthesized using 1 μM AS1411 aptamer and 1% w/v HAuCl₄ with 15-nm, 30-nm, and 50-nm gold nanoparticles as nanoseeds. FIGS. 24a-c are TEM images of the nanoflowers grown from 15-nm, 30-nm, and 50-nm gold nanoparticle seeds synthesized with the AS1411 aptamer under the conditions shown in Table 2. The protocol described above in Example 1 was followed during synthesis. As seen in FIGS. 24a-c, the nanoflower size increased with increasing nanoseed size.

TABLE 2

| | 200 μL AuNP | NH₂OH (400 mM) | 1% w/v HAuCl₄ |
|---|---|---|---|
| FIG. 24a | 15 nm, 0.5 nM | 15 μL | 3 μL |
| FIG. 24b | 30 nm, 0.31 nM | 15 μL | 3 μL |
| FIG. 24c | 50 nm, 0.06 nM | 15 μL | 4 μL |

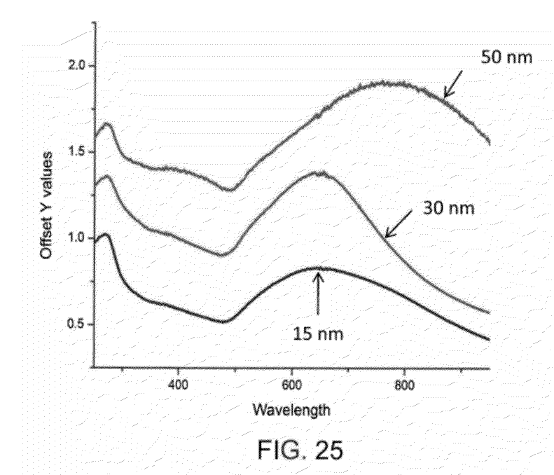

The nanoflower structure is ideally suited for photothermal applications, and embodiments of the synthesized nanoflowers can be tuned to absorb strongly within the near-IR window (i.e., from 700 nm to 900 nm). As shown in FIG. 25, the nanoflowers grown from 50-nm gold nanoparticle seeds are candidates for photothermal applications with an absorption peak at 800 nm.

### Example 9

### Cancer-Selective Targeted Uptake In Vitro

Two types of nanoflowers were synthesized. The first nanoflower included the AS1411 apatmer (SEQ ID NO: 2); the second construct was identical except the aptamer sequence was randomized (SEQ ID NO: 1). The DNA sequences are shown in Table 3 below. Both types of nanoflowers were grown from 15 nm gold seeds and incubated with MCF-7 cells (human breast cancer cells). Nanoflowers were synthesized following the protocol described above in Example 1.

Cells were incubated and grown according to standard procedures and plated on glass cover slips inside a 6-well plate (˜100,000 cells per well). Cells were incubated for 12 hours in cell medium (10% FBS) and washed with PBS buffer. After washing, the cells were incubated with 100 μL of nanoflower solution (10 nM suspended in deionized water) diluted with 900 μL of Opti-MEM for 2 hours at 37° C. and 5% CO₂. After incubation, the cells were washed 3× with PBS to remove excess nanoflowers, and the glass slides were processed for imaging under fluorescence microscope and dark-field optical microscope.

TABLE 3

| | | |
|---|---|---|
| Control- | SEQ ID NO: 1 | 5′-/56-FAM/TTG GTA GTA GTG ATT GTA ATG GTA GTG |
| DNA | | A TTTTT TTTTT TTTTT CCCCC CCCCC CCCCC CCCCC CCCCC |
| | | CCCCC-3′ |
| Aptamer- | SEQ ID NO: 2 | 5′-/56-FAM/TTG GTG GTG GTG GTT GTG GTG GTG GTG |
| DNA | | G TTTTT TTTTT TTTTT CCCCC CCCCC CCCCC CCCCC CCCCC |
| | | CCCCC-3′ |

(AS1411 aptamer sequence in bold)

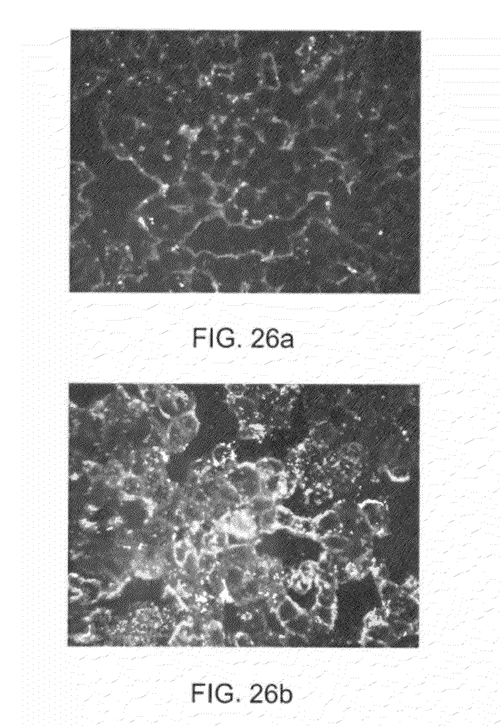

As shown in FIGS. 26a and 26b, nanoflowers functionalized with the AS1411 aptamer (FIG. 26b) exhibited superior binding to the MCF-7 cells compared to nanoflowers comprising control DNA (FIG. 26a).

### Example 10

### Diagnostic Imaging with Shaped Nanoparticles

Embodiments of the disclosed shaped nanoparticles (e.g., nanoflowers, nanoplates, nanostars, nanopeanuts, etc.) may be used for diagnostic imaging, such as to visualize the location and/or size of a tumor. For example, gold nanoflowers can be synthesized as described in Example 1. An antibody that recognizes an antigen on a tumor cell may be conjugated to the AuNFs by any suitable method. Tumor-specific antibodies are well known in the art. Alternatively, small molecules that specifically bind to tumor antigens can be used instead of antibodies. In one example, an aptamer specific for cancer cells is used.

Exemplary antibodies and small molecules that can be conjugated to the disclosed nanoparticles are provided in Table 4.

TABLE 4

| | | Exemplary |
|---|---|---|
| Tumor-Specific | | Antibody/Small |
| Antigen | Exemplary Tumors | Molecules |
| HER1 | adenocarcinoma | Cetuximab, |
| | | panitumamab, |
| | | zalutumumab, |
| | | nimotuzumab, |
| | | matuzumab. Small |
| | | molecule inhibitors |
| | | gefitinib, erlotinib, and |
| | | lapatinib can also be used. |
| HER2 | breast cancer, ovarian | Trastuzumab |
| | cancer, stomach cancer, | (Herceptin ®), pertuzumab |
| | uterine cancer | |
| CD25 | T-cell lymphoma | Daclizumab (Zenapax) |
| CEA | colorectal cancer, some | CEA-scan (Fab fragment, |
| | gastric cancers, biliary | approved by FDA), |
| | cancer | colo101 |
| Cancer antigen | ovarian cancer, | OC125 monoclonal |
| 125 (CA125) | mesothelioma, breast | antibody |
| | cancer | |
| Alpha- | hepatocellular carcinoma | ab75705 (available from |
| fetoprotein (AFP) | | Abcam) and other |
| | | commercially available |
| | | AFP antibodies |
| Lewis Y | colorectal cancer, biliary | B3 (Humanized) |
| | cancer | |
| TAG72 | adenocarcinomas | B72.3 (FDA-approved |
| | including colorectal, | monoclonal antibody) |
| | pancreatic, gastric, | |
| | ovarian, endometrial, | |
| | mammary, and non-small | |
| | cell lung cancer | |

The antibody (or small molecule) may be conjugated to the gold surface or to a DNA oligomer. The antibody-AuNF conjugates may then be administered to a subject using routine methods, for example by injection (for example intratumorally or i.v.). After waiting for a period of time sufficient to allow the conjugates to travel to and bind to the tumor cell antigens, the conjugates may be visualized by CT or x-ray imaging, thus permitting visualization of the tumor.

Alternatively, an antibody that recognizes a tumor cell antigen may be prepared. A second antibody that recognizes the anti-antigen antibody may be conjugated to the AuNFs. The anti-antigen antibody and the antibody-AuNF conjugates may be administered sequentially or simultaneously to the subject. After waiting for a period of time sufficient to allow the anti-antigen antibody to bind to the tumor cell antigen, and the antibody-AuNF conjugates to bind to the anti-antigen antibody, the conjugates may be visualized by CT or x-ray imaging.

In some examples, the antibody-AuNF conjugates are used to image tumor cells ex vivo. For example, tumor cells from a subject can be obtained (for example during a biopsy), and then incubated with the antibody-AuNF conjugates under conditions that permit the antibody to bind to its target protein. IN some examples live cells are incubated with the antibody-AuNF conjugates, while in other examples killed or fixed cells are incubated with the antibody-AuNF conjugates. The cells can be processed for imaging (for example fixed and embedded), for example using electron microscopy.

### Example 11

### Photothermal Therapy with Shaped Nanoparticles

Embodiments of the disclosed shaped nanoparticles (e.g., nanoflowers, nanoplates, nanostars, nanopeanuts, etc.) may be delivered to a target cell of interest for use in photothermal therapy. A shaped nanoparticle of a particular size and shape may be selected based on its absorbance of energy within a given wavelength range, e.g., near-infrared radiation. In certain embodiments, the shaped nanoparticle is conjugated to a moiety capable of recognizing and binding to the target cell. Suitable moieties include but are not limited to antibodies and fragments thereof, drug molecules, proteins, peptides, and aptamers.

In one example, AuNF conjugates may be delivered to tumor cells by the methods outlined in Example 10. Suitable AuNF doses may range from 20 mg per kg body weight to 20 g per kg body weight. Because AuNF conjugates are capable of absorbing near-infrared (NIR) radiation, the tumor site may be irradiated with NIR radiation (700 nm-1500 nm), such as from an NIR laser. For example, a red laser that emits light with a wavelength of 790 to 820 nm or 800 nm to 810 nm (such as 800 nm or 810 nm) may be used. In one example, the tumor is irradiated at a dose of at least 0.5 W/cm² for 2 to 60 minutes, for example 5 to 30 minutes or 3 to 10 minutes, such as at least 2 W/cm² for 2 to 60 minutes, for example 5 to 30 minutes or 3 to 10 minutes, at least 10 W/cm² for 2 to 60 minutes, for example 5 to 30 minutes or 3 to 10 minutes, or 0.5 to 50 W/cm² for 2 to 60 minutes, for example 5 to 30 minutes or 3 to 10 minutes. The tumor cells may be destroyed via photothermal heating caused when the AuNFs absorb energy from the laser.

### Example 12

### Drug Delivery with Shaped Nanoparticles

Embodiments of the disclosed shaped nanoparticles (e.g., nanoflowers, nanoplates, nanostars, nanopeanuts, etc.) may be utilized to deliver a therapeutic drug molecule to a subject. For example, AuNFs can be synthesized as described in Example 1. Therapeutic drug molecules may be conjugated to the AuNFs by any suitable means. The drug molecule may be conjugated to the gold surface or to a DNA oligomer. The drug-AuNF conjugate may then be administered to a subject as described above at a therapeutically effective dose. The drug-AuNF conjugates may be taken up by cells (e.g., by endocytosis or receptor-mediated endocytosis), thereby delivering drug to the cell interior. In one example, the drug is a chemotherapeutic agent, and is administered to a subject in order to treat a tumor in the subject.

Alternatively, the drug-AuNF conjugate may further be conjugated to an antibody that recognizes an antigen on a target cell. The drug-AuNF-antibody conjugate may be administered to a subject. The antibody may then bind to the target cell antigen, thereby delivering the drug to the immediate vicinity of the target cell while minimizing drug delivery to non-target cells.

### REFERENCES

(1) Murray, C. B.; Sun, S. H.; Doyle, H.; Betley, T. Mrs Bulletin 2001, 26, 985-991.
(2) Jin, R. C.; Cao, Y. W.; Mirkin, C. A.; Kelly, K. L.; Schatz, G. C.; Zheng, J. G. Science 2001, 294, 1901-1903.
(3) Jana, N. R.; Gearheart, L.; Murphy, C. J. J. Phys. Chem. B 2001, 105, 4065-4067.
(4) Sun, Y. G.; Xia, Y. N. Science 2002, 298, 2176-2179.
(5) Daniel, M. C.; Astruc, D. Chem. Rev. 2004, 104, 293-346.
(6) Sonnichsen, C.; Reinhard, B. M.; Liphardt, J.; Alivisatos, A. P. Nat. Biotechnol. 2005, 23, 741-745.
(7) Huang, X. H.; El-Sayed, I. H.; Qian, W.; El-Sayed, M. A. J. Am. Chem. Soc. 2006, 128, 2115-2120.
(8) Tao, A. R.; Habas, S.; Yang, P. D. Small 2008, 4, 310-325.
(9) Xia, Y.; Xiong, Y.; Lim, B.; Skrabalak, S. E. Angew. Chem., Int. Ed. 2009, 48, 60-103.
(10) Seeman, N. C.; Nature 2003, 421, 427-431.
(11) Rothemund, P. W. K.; Nature 2006, 440, 297-302;
(12) Lu, J; Liu. J; Acc. Chem. Res. 2007, 40, 315-323.
(13) Wang, Z; Y. Lu, J. Mater. Chem. 2009, 19, 1788-1798.
(14) Braun, E.; Eichen, Y.; Sivan, U.; Ben-Yoseph, G. Nature 1998, 391, 775-778.
(15) Gu, Q.; Cheng, C. D.; Gonela, R.; Suryanarayanan, S.; Anabathula, S.; Dai, K.; Haynie, D. T. Nanotechnology 2006, 17, R14-R25.
(16) Alivisatos, A. P.; Johnsson, K. P.; Peng, X.; Wilson, T. E.; Loweth, C. J.; Bruchez Jr, M. P.; and Schultz, P. G; Nature 1996, 382, 609-611.
(17) Mirkin, C. A.; Letsinger, R. L.; Mucic, R. C.; Storhoff, J. J. Nature 1996, 382, 607-609.
(18) Warner, M. G.; Hutchison, J. E. Nat. Mater. 2003, 2, 272-277.
(19) Le, J. D.; Pinto, Y.; Seeman, N. C.; Musier-Forsyth, K.; Taton, T. A.; Kiehl, R. A. Nano Lett. 2004, 4, 2343-2347;
(20) Zhang, J.; Liu, Y.; Ke, Y.; Yan, H.; Nano Lett. 2006, 6, 248-251;
(21) Lee, J. H.; Wernette, D. P.; Yigit, M. V.; Liu, J.; Wang, Z; Lu, Y. Angew. Chem., Int. Ed. 2007, 46, 9006.
(22) Bigham, S. R.; Coffer, J. L. J. Phys. Chem. 1992, 96, 10581-10584.
(23) Ma, N.; Dooley, C. J.; Kelley, S. O. J. Am. Chem. Soc. 2006, 128, 12598-12599.
(24) Kumar, A.; Jakhmola, A. Langmuir 2007, 23, 2915-2918.
(25) Ma, N.; Yang, J.; Stewart, K. M.; Kelley, S. O. Langmuir 2007, 23, 12783-12787.
(26) Berti, L.; Burley, G. A. Nat. Nanotechnol. 2008, 3, 81-87.
(27) Wang, Q. B.; Liu, Y.; Ke, Y. G.; Yan, H. Angew. Chem., Int. Ed. 2008, 47, 316-319.
(28) Ma, N.; Sargent, E. H.; Kelley, S. O, Nat. Nanotechnol. 2009, 4, 121-125.
(29) Brown, S.; Sarikaya, M.; Johnson, E. J. Mol. Biol. 2000, 299, 725-735.
(30) Naik, R. R.; Stringer, S. J.; Agarwal, G.; Jones, S. E.; Stone, M. O, Nat. Mater. 2002, 1, 169-172.
(31) Banerjee, I. A.; Yu, L. T.; Matsui, H. Proc. Natl. Acad. Sci. U.S.A. 2003, 100, 14678-14682.
(32) Dickerson, M. B.; Sandhage, K. H.; Naik, R. R. Chem. Rev. 2008, 108, 4935-4978.
(33) Storhofff, J. J.; Elghanian, R.; Mirkin, C. A.; Letsinger, R. L. Langmuir 2002, 18, 6666-6670.
(34) Ostblom, M.; Liedberg, B.; Demers, L. M.; Mirkin, C. A. J. Phys. Chem. B 2005, 109, 15150-15160.
(35) Brown, K. R.; Natan, M. J. Langmuir 1998, 14, 726-728.
(36) Bakr, O. M.; Wunsch, B. H.; Stellacci, F. Chem. Mater. 2006, 18, 3297-3301.
(37) Poon, K.; Macgregor, R. B. Biopolymers 1998, 45, 427-434.
(38) Li, H. X.; Rothberg, L. J. J. Am. Chem. Soc. 2004, 126, 10958-10961.
(39) Li, H. X.; Rothberg, L. Proc. Natl. Acad. Sci. U.S.A. 2004, 101, 14036-14039.
(40) Zhao, L.; Ji, X.; Sun, X.; Li, J.; Yang, W.; Peng, X. J. Phys. Chem. C. 2009, 113, 16645-16651.
(41) Katz, E.; Willner, I. Angew. Chem., Int. Ed. 2004, 43, 6042-6108.
(42) Demers, L. M.; Mirkin, C. A.; Mucic, R. C.; Reynolds, R. A.; Letsinger, R. L.; Elghanian, R.; Viswanadham, G. Anal. Chem. 2000, 72, 5535-5541.
(43) Kannan, B.; Kulkarni, R. P.; Majumdar, A. Nano Lett. 2004, 4, 1521-1524.
(44) Nie, S.; Emory, S. R., Science 1997, 275, 1102-1106.
(45) Hao, E. C.; Bailey, R. C.; Schatz, G. G.; Hupp, J. T.; Li, S. Y. Nano Lett. 2004, 4, 327-330.
(46) Bakr, O. M.; Wunsch, B. H.; Stellacci, F. Chem. Mater. 2006, 18, 3297-3301.
(47) Wax, A.; Sokolov, K. Laser & Photon. Rev. 2009, 3, 146-158.
(48) Giljohann, D. A.; Seferos, D. S.; Patel, P. C.; Millstone, J. E.; Rosi, N. L.; Mirkin, C. A. Nano Lett. 2007, 7, 3818-3821.
(49) Chithrani, B. D.; Ghazani, A. A.; Chan, W. C. W. Nano Lett. 2006, 6, 662-668.
(50) Millstone, J. E.; Wei, W.; Jones, M. R.; Yoo, Y; Mirkin, C. A.; Nano. Lett. 2008, 8, 2526-2569.
(51) Newman, J. D. S.; Blanchard G. J. J. Nanoparticle Res. 2007, 9, 861-868.
(52) Liu Q.; et al., Mater. Sci. 2006, 41, 3657-3662.
(53) Popovtzer, R., et al., Nano. Lett. 2008, 8, 4593-4596.
(54) Hainfeld, J. et al., J. Pharmacy and Pharmacology 2008, 60, 977-985.
(55) Liu, J.; Lu, Y., Nat. Protoc. 2006, 1, 246.
(56) Demers, L. M., et al., Anal. Chem. 2000, 72, 5535.
(57) Farokhzad et al., Proc. Natl. Acad. Sci. U.S.A. 2006, 103, 6315.
(58) Giljohann et al., Nano Lett. 2007, 7, 3818.
(59) Hauck, T. S. et al., Small 2008, 4, 153.
(60) Jain et al., J. Phys. Chem. B 2006, 110, 7238.
(61) O'Neal et al., Cancer Letters 2004, 209, 171.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. A method for controlling the shape of a nanoparticle, comprising:
providing a metal nanoseed;
adsorbing a plurality of nucleic acid oligomers to the metal nanoseed and
depositing metal onto the metal nanoseed to produce a shaped nanoparticle, wherein the shaped nanoparticle has a shape determined at least in part by the nucleic acid sequence of the nucleic acid oligomer.

2. The method of claim 1, where the metal nanoseed is gold.

3. The method of claim 2, further comprising coating the metal nanoseed with citrate before adsorbing the nucleic acid oligomer.

4. The method of claim 1 where the metal nanoseed is a nanosphere, a nanorod, or a nanoprism.

5. The method of claim 1 where the metal nanoseed has a largest dimension ranging from 1 nm to 1000 nm.

6. The method of claim 1 where each of the plurality of nucleic acid oligomers comprises a DNA sequence selected from poly A, poly C, poly G, poly T, or a sequence with mixed nucleotides of A, C, G, and/or T.

7. The method of claim 1 where each of the plurality of nucleic acid oligomers comprises an RNA sequence selected from poly A, poly C, poly G, poly U, or a sequence with mixed nucleotides of A, C, G, and/or U.

8. The method of claim 1 where each of the plurality of nucleic acid oligomers comprises an aptamer.

9. The method of claim 1 where each of the plurality of nucleic acid oligomers has 5 to 100 nucleotides.

10. The method of claim 1 where each of the plurality of nucleic acid oligomers has the same nucleic acid sequence.

11. The method of claim 1 wherein:
the metal nanoseed is a gold nanosphere, each of the plurality of nucleic acid oligomers has a DNA sequence consisting of poly A, poly C, or a mixture of A and C, and depositing gold onto the gold nanosphere produces a nanoflower; or
the metal nanoseed is a gold nanosphere, each of the plurality of nucleic acid oligomers has a DNA sequence consisting of poly T, and depositing gold onto the gold nanosphere produces a spherical nanoparticle; or
the metal nanoseed is a gold nanoprism, each of the plurality of nucleic acid oligomers has a DNA sequence consisting of poly T, poly G, or a mixture of T and G, and depositing gold onto the gold nanoprism produces a nanostar; or
the metal nanoseed is a gold nanoprism, each of the plurality of nucleic acid oligomers has a DNA sequence consisting of poly A, poly C, or a mixture of A and C, and depositing gold onto the gold nanoprism produces a nanoplate.

12. The method of claim 1 where at least one of the plurality of nucleic acid oligomers is labeled with a detectable label.

13. A shaped nanoparticle made by the method of claim 1.

14. A shaped nanoparticle, comprising:
a metal nanoparticle; and
a plurality of nucleic acid oligomers extending from the metal nanoparticle, wherein at least a portion of each of the plurality of nucleic acid oligomers is embedded within the metal nanoparticle.

15. The shaped nanoparticle of claim 14 where each of the plurality of nucleic acid oligomers is 5 to 100 nucleotides in length.

16. The shaped nanoparticle of claim 14 where the metal nanoparticle is gold.

17. The shaped nanoparticle of claim 14 wherein:
each of the nucleic acid oligomers has a DNA sequence consisting of poly A, poly C, or a mixture of A and C, and the shaped nanoparticle is a nanoflower or a nanoplate; or
each of the nucleic acid oligomers has a DNA sequence consisting of poly T, poly G or a mixture of T and G, and the shaped nanoparticle is a nanosphere or a nanostar; or
each of the nucleic acid oligomers has an RNA sequence consisting of poly A, poly C, poly U, poly G, or a mixture of A, C, U, and/or G.

18. The shaped nanoparticle of claim 14 where each of the nucleic acid oligomers comprises an aptamer.

19. A method of delivering a shaped nanoparticle to a target cell, comprising:
providing a shaped nanoparticle comprising a metal nanoparticle and a plurality of nucleic acid oligomers extending from the metal nanoparticle, wherein at least a portion of each of the plurality of nucleic acid oligomers is embedded within the metal nanoparticle; and
contacting the shaped nanoparticle with a target cell under conditions that allow the shaped nanoparticle to bind to and/or enter the cell, wherein the shaped nanoparticle comprises an antibody specific for a protein on the surface of the target cell, thereby delivering the shaped nanoparticle to a target cell.

20. The method of claim 19, further comprising imaging the shaped nanoparticle.

21. The method of claim 19 where the target cell is in a subject, and contacting comprises administering the shaped nanoparticle to the subject.

22. The method of claim 21, further comprising administering near-infrared radiation to the subject, wherein the shaped nanoparticle absorbs at least a portion of the near-infrared radiation, thereby producing a temperature increase within the shaped nanoparticle.

23. A method of delivering a drug within a cell, comprising:
providing a shaped nanoparticle-drug conjugate comprising
a metal nanoparticle,
a plurality of nucleic acid oligomers extending from the metal nanoparticle, wherein at least a portion of each of the plurality of nucleic acid oligomers is embedded within the metal nanoparticle, and
a drug molecule conjugated to the shaped nanoparticle to produce the shaped nanoparticle-drug conjugate; and
contacting the shaped nanoparticle with the cell, wherein the shaped nanoparticle-drug conjugate is contacted with the cell under conditions sufficient to allow the cell to internalize the shaped nanoparticle-drug conjugate.

24. The method of claim 23, wherein the cell is in a subject, and contacting comprises administering a therapeutic amount of the shaped nanoparticle-drug conjugate to the subject.
